(19) United States
(12) Patent Application Publication
(10) Pub. No.: US 2020/0405884 A1
(43) Pub. Date: Dec. 31, 2020

(54) CRISPR-NANOPARTICLES AND METHODS OF USE IN BRAIN DISORDERS

(71) Applicant: Board of Regents, The University of Texas System, Austin, TX (US)

(72) Inventors: Hye Young Lee, San Antonio, TX (US); Bumwhee Lee, San Antonio, TX (US); Kunwoo Lee, Berkeley, CA (US); Hyo Min Park, Berkeley, CA (US)

(21) Appl. No.: 16/979,046

(22) PCT Filed: Mar. 8, 2019
(86) PCT No.: PCT/US2019/021372

§ 371 Date: Sep. 8, 2020

Related U.S. Application Data
(60) Provisional application No. 62/641,099, filed on Mar. 9, 2018.
(60) Provisional application No. 62/678,140, filed on May 30, 2018.

(51) Int. Cl. A61K 48/00; C12N 9/22; C08K 3/08

(52) U.S. Cl. CPC A61K 48/0041; B82Y 5/00; C08K 3/08; C12N 9/22

(57) ABSTRACT

Described herein are compositions and methods for treating diseases and disorders of the brain using a non-viral nanoparticle delivery of CRISPR. Disclosed herein are compositions comprising CRISPR-Gold compositions comprising DNA oligonucleotides, RNA-directed nucleases and guide RNAs. The methods include modulating expression of a gene in a cell using said compositions, inducing site-specific DNA cleavage in a cell, and treating a subject having fragile X syndrome caused by increased metabotropic glutamate receptor 5 signaling using the compositions disclosed herein.

### CROSS REFERENCE TO RELATED APPLICATIONS

This application claims the benefit of the filing dates of U.S. Provisional Application No. 62/641,099, filed on Mar. 9, 2018; and U.S. Provisional Application No. 62/678,140, filed on May 30, 2018. The content of these earlier filed applications is hereby incorporated by reference in their entirety.

### REFERENCE TO A SEQUENCE LISTING

The Sequence Listing submitted herein as a text file named “21105_0063P1_SL.txt,” created on Mar. 7, 2019, and having a size of 8,192 bytes is hereby incorporated by reference pursuant to 37 C.F.R. § 1.52(e)(5).

### BACKGROUND

Programmable RNA-guided endonucleases have the potential to revolutionize the treatment of neurological diseases because of their ability to cut genes with sequence specificity (Jinek, M. et al. Science 337, 816-821 (2012); Cong, L. et al. Science 339, 819-823 (2013); Mali, P. et al. Science 339, 823-826 (2013); Cho, S. W., Kim, S., Kim, J. M. & Kim, J. S. Nat Biotechnol 31, 230-232 (2013); and Zetsche, B. et al. Cell 163, 759-771 (2015)). Despite their potential, the translational impact of RNA-guided endonucleases on the central nervous system has been limited due to challenges in performing efficient gene editing in adult brains with minimal toxicity. Currently, gene editing in the adult brain is mainly accomplished by viral delivery of CRISPR-Cas9 (Swiech, L. et al. Nat Biotechnol 33, 102-106 (2015)). However, the translation of viral delivery methods for CRISPR-Cas9 and sgRNA in the brain can be challenging because of the immunogenicity of viruses (Mingozzi, F. & High, K. A. Blood 122, 23-36 (2013)), the genomic damage caused from the prolonged expression of CRISPR-Cas9 and sgRNA (Ishida, K., Gee, P. & Hotta, A. Int J Mol Sci 16, 24751-24771 (2015)), and the toxicity caused from the continuous expression of foreign proteins in neurons, which frequently causes changes in neuronal phenotypes (Watakabe, A. et al. Neurosci Res 93, 144-157 (2015)). Therefore, there is a need to develop new methods for delivering RNA-guided endonucleases into the brain of adult mice.

### SUMMARY

Disclosed herein, are CRISPR-Gold systems comprising: a) a plurality of DNA oligonucleotides conjugated to a gold nanoparticle forming a DNA oligonucleotide-gold nanoparticle (GNP); b) one or more RNA-guided endonuclease proteins conjugated to one or more guide RNA molecules (gRNA) forming one or more ribonucleoprotein (RNPs); and c) a biodegradable polymer.

Disclosed herein, are methods of modulating expression of a gene in a cell, the methods comprising: a) introducing into the cell a CRISPR-Gold system, comprising: i) a plurality of DNA oligonucleotides conjugated to a gold nanoparticle forming a DNA oligonucleotide-gold nanoparticle (GNP); ii) one or more RNA-guided endonuclease proteins conjugated to one or more guide RNA molecules (gRNA) forming one or more ribonucleoprotein (RNPs), wherein the gRNA is complementary to a target nucleic acid sequence comprising the gene; and iii) a biodegradable polymer.

Disclosed herein, are methods for introducing into a cell a CRISPR-Gold system comprising: a) a plurality of DNA oligonucleotides conjugated to a gold nanoparticle forming a DNA oligonucleotide-gold nanoparticle (GNP); b) one or more RNA-guided endonuclease proteins conjugated to one or more guide RNA molecules (gRNA) forming one or more ribonucleoprotein (RNPs); wherein the gRNA hybridizes with a target sequence of a DNA molecule in a cell; and c) a biodegradable polymer.

Disclosed herein, are methods of treating a subject having fragile X syndrome, the methods comprising: (a) determining mGluR 5 signaling or a mGluR5-mediated behavioral phenotype in the subject; (b) administering to the subject a pharmaceutical composition comprising a CRISPR-Gold system comprising one or more RNA-guided endonuclease proteins conjugated to one or more guide RNA molecules (gRNA), wherein the gRNA can be SEQ ID NO: 36.

Disclosed herein, are methods for targeted genomic modification of Grm5 in mammalian cells, the methods comprising administering a CRISPR-Gold nanoparticle, wherein the CRISPR-Gold nanoparticle comprises a guide RNA sequence that targets and hybridizes with a sequence that encodes Grm5.

Disclosed herein, are CRISPR-Gold systems for targeted genomic modification of Grm5 in mammalian cells, wherein the systems comprise a guide RNA sequence that targets and hybridizes with a sequence that encodes Grm5.

Other features and advantages of the present compositions and methods are illustrated in the description below, the drawings, and the claims.

### BRIEF DESCRIPTION OF THE DRAWINGS

FIGS. 1A-F show that there is no significant physiological deficit nor cytotoxicity found in primary cultured neurons after CRISPR-Gold treatment. FIG. 1A is a schematic diagram of CRISPR-Gold synthesis. FIGS. 1B-D shows the days in vitro 7 (DIV7) primary cultured neurons were treated with CRISPR-Gold Cas9 RNPs (CRISPR-Gold) and were compared with untreated neurons (Control) for electrophysiological properties by whole-cell current clamp recording. Neurons were measured with (B) membrane potentials (mV), (C) input resistance (GOHms), and (D) the number of spikes generated by a 200 pA current injection. n=21 for Control, n=17 for CRISPR-Gold, mean±SEM. FIG. 1E shows representative traces for Control and CRISPR-Gold. FIG. 1F shows DIV7 primary cultured neurons treated with CRISPR-Gold Cas9 RNPs (CRISPR-Gold) (Left panels) that were compared with untreated neurons (Control). Scale bar, 100 μm. Right panels: Quantification of SYTOX⁺ cells (%) among DAPI⁺ cells in Control or CRISPR-Gold group. n=6 for each group, mean±SEM. No significant difference in the % of SYTOX⁺ cells was found between groups. This experiment was replicated twice.

FIGS. 2A-D shows YFP expression is efficiently reduced in the neurons of the mouse brain using CRISPR-Gold delivery of Cas9 or Cpf1 RNPs in Thy1-YFP mice. FIG. 2A is a schematic showing CRISPR-Gold delivery of Cas9 or Cpf1 RNPs into the brains of Thy1-YFP mice. FIG. 2B is a schematic of Cas9 or Cpf1 RNP-mediated indel mutation and the stereotaxic injection into the hippocampus (Bregma: −2.18 mm) of Thy1-YFP mice using the CRISPR-Gold system. FIGS. 2C-D shows the immunostaining (left panels of YFP-labeled neurons (green) with nuclei staining with DAPI (blue) 2 weeks after stereotaxic injection of (c) Cas9 or (d) Cpf1 RNPs using the CRISPR-Gold system into the dentate gyrus of the hippocampus of Thy1-YFP mice, scale bar, 100 μm; right panels: Quantification of the YFP⁺ cells normalized to DAPI⁺ cell number in the granule cell layer of the dentate gyms in the injected side (CRISPR-Gold) compared to the contralateral control side (Control). n=9, mean±SEM, ****P<0.0001 as compared to the Control side, Student's unpaired t-test. This experiment was replicated four times.

FIGS. 3A-D shows that deletion of stop sequences and expression of tdTomato in the brain of Ai9 mice by CRISPR-Gold delivery of Cas9 or Cpf1 RNPs into the hippocampus.

FIG. 3A is a schematic of CRISPR-Gold delivery of Cas9 or Cpf1 RNPs into the brains of Ai9 mice. FIG. 3B is a schematic of Cas9 or Cpf1 RNP-mediated deletion and the stereotaxic injection into the hippocampus (Bregma: −2.18 mm) of Ai9 mice using the CRISPR-Gold system. FIGS. 3C-D shows immunostaining of tdTomato (red) and nuclei staining with DAPI (blue) 2 weeks after stereotaxic injection of (c) Cas9 RNPs or (d) Cpf1 RNPs using the CRISPR-Gold system into the hippocampus of Ai9 mice (left panels); and the uninjected side (Control) and the injected side (CRISPR-Gold) are shown in the upper panels. Scale bar, 200 μm. Higher magnification images of the injected side (yellow box) are shown in the lower panels. Scale bar, 100 μm. Right panels: Quantification of the % of tdTomato⁺ cells among DAPI⁺ cells in the (c) Cas9 RNP-injected area. n=12, mean±SEM, and the (d) Cpf1 RNP-injected area. n=12, mean±SEM, ****P<0.0001 as compared to the Control side, Student's unpaired t-test. This experiment was replicated twice.

FIGS. 4A-E shows deletion of stop sequences and expression of tdTomato in the brain of Ai9 mice by CRISPR-Gold delivery of Cas9 or Cpf1 RNPs into the striatum. FIG. 4A is a schematic of Cas9 or Cpf1 RNP-mediated deletion and the stereotaxic injection into the striatum (Bregma: 0.26 mm) of Ai9 mice using the CRISPR-Gold system. FIGS. 4B-C shows immunostaining of tdTomato (red) and nuclei staining with DAPI (blue) 2 weeks after stereotaxic injection of (b) Cas9 RNPs or (c) Cpf1 RNPs using the CRISPR-Gold system into the striatum of Ai9 mice (left panels). The uninjected side (Control) and the injected side (CRISPR-Gold) are shown in the upper panels. Scale bar, 400 μm. Higher magnification images of the injected side (yellow box) are shown in the lower panels. Scale bar, 200 μm. Right panels: Quantification of the % of tdTomato⁺ cells among DAN⁺ cells in the (b) Cas9 RNP-injected area. n=14, mean±SEM, and (c) Cpf1 RNP-injected area. n=14, mean±SEM, ****P<0.0001 as compared to the Control side, Student's unpaired t-test. This experiment was replicated twice. FIGS. 4D-E shows the quantification of the number of DAPI⁺ cells in the (d) Cas9 or (e) Cpf1 RNP-injected area (striatum). n=6, mean±SEM, ns=non-significant difference, Student's unpaired t-test. This experiment was replicated twice.

FIGS. 5A-C show that mGluR5-CRISPR successfully promotes mGluR5 gene editing in the striatum of WT or Fmr1 KO mice. FIG. 5A is a schematic of injection for mGluR5-CRISPR into the striatum of WT or Fmr1 KO mice. Saline or mGluR5-CRISPR was injected into the striatum (Bregma: 0.26 mm, 3 injection sites per hemisphere are indicated as blue dots, 0.4 mm interval) of WT or Fmr1 KO mice. Schematic design and the target sequences of Cas9 RNPs for mGluR5 gene (Grm5) knockout are shown (SEQ ID NOs: 27 and 28). FIG. 5B shows that RNA was extracted from the saline-injected control side (Control) or from the mGluR5-CRISPR-injected side (mGluR5-CRISPR) of WT or Fmr1 KO mice 11 weeks after stereotaxic injections. mRNA levels of mGluR5 were amplified and analyzed by running RT-qPCR. Fold change of mGluR5 mRNA levels are shown after normalization with PPIA mRNA levels. n=4-6, mean±SEM, ***P<0.001, ****P<0.0001, one-way ANOVA. FIG. 5C shows immunostaining of mGluR5 (cyan) 5 weeks after stereotaxic injection of saline (Control) or mGluR5-CRISPR (mGluR5-CRISPR) into the striatum of WT or Fmr1 KO mice (left panels) and the results of quantification of the number of mGluR5⁺ cells in WT Control, WT mGluR5-CRISPR, Fmr1 KO Control, and Fmr1 KO mGluR5-CRISPR, were counted and normalized to the number of DAPI⁺ cells (right panels). n=8-10, mean±SEM, ****P<0.0001 by one-way ANOVA. P values were calculated between WT Control and Fmr1 KO Control, WT Control and WT mGluR5-CRISPR, or Fmr1 KO Control and Fmr1 KO mGluR5-CRISPR.

FIGS. 6A-B shows that knocking out mGluR5 using mGluR5-CRISPR significantly rescues the increased repetitive behaviors in Fmr1 KO mice. Three weeks after stereotaxic injection of either saline (Control) or mGluR5-CRISPR (mGluR5-CRISPR) into the striatum of WT and Fmr1 KO mice, (a) the marble bury assay or (b) the empty cage observation test was performed. FIG. 6A shows: Percentage of marbles buried after 30 min of the marble bury test (left panel) and representative images after the marble bury assay for 30 min (right panel). FIG. 6B show that jumping behavior (left panel) and line crossing behavior (right panel) were scored during 10 min of an empty cage observation test. For FIGS. 6A-B: n=11, 12, 12, and 12 for WT Control, WT mGluR5-CRISPR, Fmr1 KO Control, and Fmr1 KO mGluR5-CRISPR respectively, mean±SEM, *P<0.05, **P<0.01, ***P<0.001 by one-way ANOVA. P values were calculated between WT Control and Fmr1 KO Control, WT Control and WT mGluR5-CRISPR, or Fmr1 KO Control and Fmr1 KO mGluR5-CRISPR.

FIGS. 7A-B shows that loading RNPs on CRISPR-Gold is verified in vitro. FIG. 7A shows sgRNA/Cas9 loading on CRISPR-Gold. FIG. 7B shows crRNA/Cpf1 loading on CRISPR-Gold. FIGS. 7A-B also shows CRISPR-Gold particles before and after the purification step were analyzed in a polyacrylamide gel.

FIGS. 8A-D show yfp knockout by Cas9 RNPs and Cpf1 RNPs are verified in YFP-HEK cells and Thy1-YFP mice. FIG. 8A is a schematic design of Cas9 RNPs and Cpf1 RNPs for yfp knockout in Thy1-YFP mice. Target sequences of Cas9 RNPs (SEQ ID NOs: 29 and 30) and Cpf1 RNPs (SEQ ID NOs: 31 and 32) are shown. FIG. 8B shows yfp knockout frequency in YFP-expressing HEK (YFP-HEK) cells after Cas9 or Cpf1 delivery. yfp knockout frequency was quantified by flow cytometry 7 days after Cas9 and Cpf1 treatments on YFP-HEK cells. FIGS. 8C-D show frequent mutations of YFP gene editing using (c) Cas9 or (d) Cpf1 in the Thy1-YFP mouse brain.

FIGS. 9A-B show that YFP intensity in the molecular layer of the dentate gyrus in CRISPR-Gold-injected mice is reduced. YFP intensity of the dentate gyrus in the hippocampus of Thy1-YFP mice was measured after injecting (a) Cas9 RNPs or (b) Cpf1 RNPs using the CRISPR-Gold system. n=9, mean±SEM, ***P<0.001, ****P<0.0001 as compared to the Control side, Student's unpaired t-test. This experiment was replicated four times.

FIGS. 10A-B show deletion of stop sequences and expression of tdTomato using Cas9 RNPs and Cpf1 RNPs are verified in Ai9-driven cells using CRISPR-Gold. FIG. 10A is a schematic design of Cas9 RNPs and Cpf1 RNPs for deletion in Ai9 mice. FIG. 10B shows the expression of tdTomato after gene editing was confirmed in primary cultured fibroblasts from Ai9 mice.

FIGS. 11A-D shows the results of an analysis of the cell type-specific effects of Cas9 or Cpf1 RNPs using CRISPR-Gold delivery into the hippocampus. FIGS. 11A, C show immunostaining of tdTomato (red) and either GFAP (cyan), Iba1 (gray), or NeuN (green) 2 weeks after stereotaxic injection of (a) Cas9 or (c) Cpf1 RNPs using the CRISPR-Gold system into the hippocampus of Ai9 mice. Scale bar, 100 μm. FIGS. 11, D shows the quantification of the GFAP⁺, Iba1⁺, or NeuN⁺ cells among the tdTomato⁺ cells (%) respectively in the (b) Cas9 RNP- or (d) Cpf1 RNP-injected area (left panel); and the quantification of the GFAP⁺, Iba1⁺, or NeuN⁺ with tdTomato⁺ cells among the total GFAP⁺, Iba1⁺ or NeuN⁺cells (%) respectively in the (b) Cas9 RNP- or (d) Cpf1 RNP-injected area (right panels). n=4 for GFAP, Iba1, or NeuN staining, mean±SEM. This experiment was replicated twice.

FIGS. 12A-D show the results of an analysis of the cell type-specific effects of Cas9 or Cpf1 RNPs using CRISPR-Gold delivery into the striatum. FIGS. 12A, C show immunostaining of tdTomato (red) and either GFAP (cyan), Iba1 (gray), or NeuN (green) 2 weeks after stereotaxic injection of (a) Cas9 or (c) Cpf1 RNPs using the CRISPR-Gold system into the striatum of Ai9 mice. Scale bar, 100 μm. FIGS. 12B, D show quantification of the GFAP⁺, Iba1⁺, or NeuN⁺ cells among the tdTomato⁺ cells (%) respectively in the (b) Cas9 RNP- or (d) Cpf1 RNP-injected area (left panels); and quantification of the GFAP⁺, Iba1⁺, or NeuN⁺ with tdTomato⁺ cells among the total GFAP⁺, Iba1⁺ or NeuN⁺ cells (%) respectively in the (b) Cas9 RNP- or (d) Cpf1 RNP-injected area (right panels). n=6, 4, or 4 for GFAP, Iba1, or NeuN staining, mean±SEM. This experiment was replicated twice.

FIGS. 13A-B shows that gene editing of the mGluR5 gene is confirmed in vitro and in cells. FIGS. 13A-B show that Grm5 sgRNAs and Cas9 proteins are able to induce mGluR5 gene editing in vitro (in tube cleavage assay) and (b) in primary cultured myoblasts with an electroporation test. In order to confirm the activity of Grm5 Cas9 RNPs, (a) the mGluR5 gene was PCR-amplified and incubated with Grm5 sgRNAs and Cas9 (Cas9 RNPs) in vitro, and (b) electroporation was conducted with primary myoblasts from mice, then mGluR5 gene was amplified with PCR, and the Surveyor assay was conducted to check target gene editing.

FIGS. 14A-B show that the TIDE assay was performed to quantify the indel frequency, and no obvious off-target DNA damage was detected among the two predicted off-target sites of Grm5 sgRNAs. FIG. 14A shows the representative TIDE analysis for Grm5-sgRNAs. R² indicates the variance, a statistic value of likelihood of the TIDE prediction. Percent of mutant frequencies (Mut. Freq.) was calculated by the summation of all indel events (%) from -20 to 20. FIG. 14B showtTwo off-target predicted sites (OT1 and OT2) were tested with PCR and the Surveyor assay from mouse brain DNA (Ml: mouse 1 and M2: mouse 2) after mGluR5-CRISPR (CRISPR-Gold complex) injection. No obvious levels of off-target were detected.

FIGS. 15A-B show that no significant increase of the innate immune response is detected in mGluR5-CRISPR-treated brains. RNA was extracted from WT or Fmr1 KO mice of Control or mGluR5-CRISPR-injected striatum. mRNA levels of microglia markers, (a) Iba1 and (b) CX3CR1, were quantified by RT-qPCR. n=6-8, mean±SEM, One-way ANOVA. No significant difference was found between any of the groups. This experiment was replicated twice.

FIGS. 16A-B shows that CRISPR-Gold-mediated mGluR5 gene knockout in the striatum does not cause altered locomotor behaviors in Fmr1 KO mice. FIG. 10A shows that the total distance of traveling was measured by the open field activity assay for 30 min. FIG. 10B shows the latency to fall was recorded by the rotarod test. n=11, 12, 12, and 12 for WT Control, WT mGluR5-CRISPR, Fmr1 KO Control, and Fmr1 KO mGluR5-CRISPR, mean±SEM, *P<0.05, One-way ANOVA. P values were calculated between WT Control and Fmr1 KO Control, WT Control and WT mGluR5-CRISPR, and Fmr1 KO Control and Fmr1 KO mGluR5-CRISPR.

FIG. 17 shows that no significant change is detected in the body weights of saline or mGluR5-CRISPR-injected WT or Fmr1 KO mice. n=11, 12, 12, and 12 for WT Control, WT mGluR5-CRISPR, Fmr1 KO Control, and Fmr1 KO mGluR5-CRISPR, mean±SEM, One-way ANOVA. No significant difference was found between any of the groups.

### DETAILED DESCRIPTION

Many modifications and other embodiments of the present disclosure set forth herein will come to mind to one skilled in the art to which this disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosures. Further, the dates of publication provided herein can be different from the actual publication dates, which can require independent confirmation.

### DEFINITIONS

As used in the specification and in the claims, the term “comprising” can include the aspects “consisting of” and “consisting essentially of” “Comprising can also mean “including but not limited to.”

As used in the specification and the appended claims, the singular forms “a,” “an” and “the” can include plural referents unless the context clearly dictates otherwise. Thus, for example, reference to “a compound” includes mixtures of compounds; reference to “a pharmaceutical carrier” includes mixtures of two or more such carriers, and the like.

The word “or” as used herein means any one member of a particular list and also includes any combination of members of that list.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment, such as, for example, prior to the administering step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

“Inhibit,” “inhibiting” and “inhibition” mean to diminish or decrease an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100%, or any amount of reduction in between as compared to native or control levels. In an aspect, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in an aspect, the increase or promotion can be a 10, 20, 30, 40, 50, 60, 70, 80, 90, 100%, or more, or any amount of promotion in between compared to native or control levels. In an aspect, the increase or promotion is 10-20, 20-30, 30-40, 40-50, 50-60, 60-70, 70-80, 80-90, or 90-100% as compared to native or control levels. In an aspect, the increase or promotion is 0-25, 25-50, 50-75, or 75-100%, or more, such as 200, 300, 500, or 1000% more as compared to native or control levels. In an aspect, the increase or promotion can be greater than 100 percent as compared to native or control levels, such as 100, 150, 200, 250, 300, 350, 400, 450, 500% or more as compared to the native or control levels.

As used herein, the term “determining” can refer to measuring or ascertaining a quantity or an amount or a change in activity. For example, determining the amount of a disclosed polypeptide in a sample as used herein can refer to the steps that the skilled person would take to measure or ascertain some quantifiable value of the polypeptide in the sample. The art is familiar with the ways to measure an amount of the disclosed polypeptides and disclosed nucleotides in a sample.

The phrase “nucleic acid” as used herein refers to a naturally occurring or synthetic oligonucleotide or polynucleotide, whether DNA or RNA or a DNA-RNA hybrid, single-stranded or double-stranded, sense or antisense, which is capable of hybridization to a complementary nucleic acid by Watson-Crick base-pairing. Nucleic acids as disclosed herein can also include nucleotide analogs (e.g., BrdU), and non-phosphodiester internucleoside linkages (e.g., peptide nucleic acid or thiodiester linkages). In particular, nucleic acids can include, without limitation, DNA, RNA, cDNA, gDNA, ssDNA, dsDNA or any combination thereof.

As used herein, the term “complementary” refers to the ability of a nucleic acid to form hydrogen bond(s) with another nucleic acid sequence by either traditional Watson-Crick or other non-traditional types. A percent complementary indicates the percentage of residues in a nucleic acid molecule which can form hydrogen bonds (e.g., Wastson-Crick base pairing) with a second nucleic acid sequence (e.g., 5, 6, 7, 8, 9, 10 out of 10 being 50%, 60%, 70%, 80%, 90%, and 100% complementary).

The terms Cas enzyme, CRISPR enzyme, CRISPR protein Cas protein and CRISPR Cas are generally used interchangeably and can refer to Cas9 and/or Cpf1 proteins.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

A wide number of neurological diseases could be treated with gene editing-based therapeutics. Of these, fragile X syndrome (FXS) is an attractive target for gene editing-based therapeutics because of the current lack of therapeutic options and the high morbidity that this disease causes. FXS is an autism-associated single-gene mutation-based disorder, which is driven by a repeat expansion mutation in the fragile X mental retardation 1 (FMRJ) gene, which encodes the fragile X mental retardation protein (FMRP), an mRNA binding protein (Kazdoba, T. M., Leach, P. T., Silverman, J. L. & Crawley, J. N. Intractable Rare Dis Res 3, 118-133 (2014)). FXS is the most common inherited form of intellectual disability (ID) and a common single-gene form of autism spectrum disorders (ASDs), accounting for approximately 2.1% of patients (Persico, A. M. & Napolioni, V. Behav Brain Res 251, 95-112 (2013)). Current drug treatments, such as psychostimulants, antidepressants, and antipsychotics, are ineffective because they do not address the underlying etiology of FXS; they target individual symptoms (Ji, N. Y. & Findling, R. L. Curr Opin Psychiatry 29, 103-125 (2016); and Politte, L. C., Henry, C. A. & McDougle, C. J. Harv Rev Psychiatry 22, 76-92 (2014)). In addition, current FXS pharmacotherapies also cause severe side effects such as weight gain and sedation (Politte, L. C., Henry, C. A. & McDougle, C. J. Harv Rev Psychiatry 22, 76-92 (2014)). New treatments for FXS are urgently needed; however, developing FXS treatments based on traditional small molecules has been challenging because of the limited number of validated FXS therapeutic targets and the difficulties associated with developing therapeutics that can provide long term effectiveness without causing cytotoxicity to brain cells.

CRISPR-based editing of the brain, generated by a local intracranial injection, has potential for treating FXS because it can lead to localized gene editing in the brain, and would therefore spare the patient the toxic effects of globally inhibiting neuronal signaling pathways. In addition, CRISPR gene editing is permanent and would make repeated injections unnecessary, making it feasible in a variety of clinical scenarios. There are several genes that are targets for a CRISPR-based FXS therapy; of these, the metabotropic glutamate receptor 5 (mGluR5) is a target because its exaggerated signaling has been demonstrated to be associated with FXS, as well as with other ASDs (Bear, M. F., Huber, K. M. & Warren, S. T. Trends Neurosci 27, 370-377 (2004); Bear, M. F. Genes Brain Behav 4, 393-398 (2005); Dölen, G. & Bear, M. F. J Physiol 586, 1503-1508 (2008); Osterweil, E. K., Krueger, D. D., Reinhold, K. & Bear, M. F. J Neurosci 30, 15616-15627 (2010); Tao, J. et al. J Neurosci 36, 11946-11958 (2016); and Silverman, J. L. et al. Sci Transl Med 4, 131ra151 (2012)). The importance of modulating mGluR5 in ASDs triggered several pharmaceutical companies to develop small molecule-based therapies that target mGluR5; however, these small molecules failed in clinical trials (Jacquemont, S. et al. Sci Transl Med 3, 64ra61 (2011); and Raspa, M., Wheeler, A. C. & Riley, C. Pediatrics 139, S153-S171 (2017)). Therefore, knocking out the mGluR5 gene (Grm5) locally in brain regions hypothesized to cause the behavioral phenotypes in FXS patients has potential for treating FXS and autism-related disorders. For gene editing to become a therapeutic treatment for FXS it is important to understand the following: the delivery challenges associated with gene editing in the brain with Cas9 RNPs; and, if mGluR5-mediated behavioral phenotypes are caused by focal over-activation of mGluR5 signaling and, if so, which parts of the brain need to have the exaggerated mGluR5 signaling reduced to rescue from a specific behavioral phenotype.

Disclosed herein is a non-viral Cas9 delivery vehicle, termed CRISPR-Gold (Lee, K. et al. Nat Biomed Eng 1, 889-901 (2017)), to deliver the RNA-guided endonucleases Cas9 and Cpf1 into the brains of adult mice and perform gene editing using Thy1-YFP and Ai9 mice. The mGluR5 gene was targeted to reduce the exaggerated mGluR5 signaling in the striatum of the mouse model of FXS and showed that the CRISPR-Gold-mediated-mGluR5 reduction rescued striatum-dependent exaggerated repetitive behaviors measured by the marble bury assay and jumping behaviors. The results described herein demonstrate that CRISPR-Gold has the potential to significantly accelerate the development of new brain targeted therapeutics but also permit the rapid development of focal brain knockout models for mechanistic, brain region, or preclinical studies, given its ability to edit genes in adult brains with low toxicity.

Little is known about the ability of RNA-guided endonucleases to transfect the brains of adult animals via non-viral methods. Currently, there is one report of non-viral gene editing in the adult brain using an intracranial injection with Cas9 ribonucleoproteins (RNPs) engineered with multiple nuclear localization sequence (NLS) signals (Staahl, B. T. et al. Nat Biotechnol 35, 431-434 (2017)). In addition, there are no reports of non-viral gene editing in the brain with RNA-guided endonucleases outside of Cas9, making it unclear if other RNA-guided endonucleases, such as Cpf1, can perform gene editing in the adult brain via non-viral delivery methods.

The Examples disclosed herein show here that CRISPR-Gold can deliver both Cas9 and Cpf1 RNPs in the brain after an intracranial injection and can edit genes. The Examples disclosed herein also show that CRISPR-mediated gene editing in neuronal cells and in non-neuronal cells that are important for brain function, including astrocytes and microglia, which has not previously been demonstrated. Also, the results described herein show for the first time that the non-viral delivery of Cpf1 RNPs in vivo is also possible, and can result in efficient gene editing and deletion of target sequences. In addition, the data demonstrate that microglia can be edited by Cas9 and Cpf1 RNPs. Microglia are important targets in a variety of diseases, but they are difficult to target for genetic manipulation via transfection or transduction due to having similar characteristics to the macrophage (Masuda, T., Tsuda, M., Tozaki-Saitoh, H. & Inoue, K. Methods Mol Biol 1041, 63-67 (2013); Balcaitis, S., Weinstein, J. R., Li, S., Chamberlain, J. S. & Möller, T. Glia 50, 48-55 (2005); and Burke, B., Sumner, S., Maitland, N. & Lewis, C. E. J Leukoc Biol 72, 417-428 (2002)). In addition, CRISPR-Gold was able to inhibit up to 40-50% of mGluR5 expression in the striatum after an intracranial injection, and this level of inhibition was able to rescue mice from the increased repetitive behaviors, which is one of the core symptoms of ASDs. Importantly, CRISPR-Gold-mediated editing was localized to the striatum and suggests that global inhibition of neuronal signaling pathways is not needed for treating autism or other brain disorders, and provides a non-toxic methodology for treating a large variety of untreatable brain disorders. Therefore, CRISPR-Gold-mediated delivery of Cas9 and Cpf1 has numerous therapeutic applications, and may be used for treating brain disorders mediated by neuronal or non-neuronal cell dysfunctions.

Compositions

The compositions disclosed herein include a CRISPR-Gold system. The CRISPR-Gold system can be non-naturally occurring.

The CRISPR-Gold system can be made up of gold nanoparticles that are combined with oligonucleotide DNA. The oligonucleotide DNA can be combined to form a complex with the CRISPR components and a polymer. The polymer can be used to facilitate the penetration of the nanoparticle into the cell. In an aspect, the CRISPR components can be a Cas9 protein or Cpf1 protein, a guide RNA and oligonucleotide DNA. The oligonucleotide DNA can be used as a template to edit the mutant sequence to wild type.

In an aspect, the CRISPR-Gold system comprises a) a plurality of DNA oligonucleotides conjugated to a gold nanoparticle forming a DNA oligonucleotide-gold nanoparticle (GNP); b) one or more RNA-guided endonuclease proteins conjugated to one or more guide RNA molecules (gRNA) forming one or more ribonucleoprotein (RNPs); and c) a biodegradable polymer. In an aspect, the one or more RNPs can be conjugated to the GNP forming a RNP-GNP complex. In an aspect, the biodegradable polymer can encapsulate the RNP-GNP complex thereby forming the CRISPR-Gold system. In an aspect, the gRNA can hybridize with a target sequence of a DNA locus in a cell. In an aspect, the gRNA can target and hybridize with a target sequence and can direct the one or more RNA-guided endonuclease proteins to the DNA locus.

In some aspects, CRISPR-Gold system includes guide RNA (gRNA). In some aspects, the gRNA can hybridize with a target sequence of a DNA molecule or locus in a cell. The gRNA can target and hybridize with the target sequence. In some aspects, the gRNA can also direct the RNA-directed nuclease into the DNA molecule or locus. In some aspects, gRNA can be selected from the group listed in Table 4. In an aspect, the gRNA can be purified. In an aspect, the gRNA can be specific for a locus of interest. In an aspect, the gRNA can be upstream of a PAM sequence.

In some aspects, the CRISPR-Gold system includes a RNA-directed endonuclease protein. In an aspect, the RNA-directed endonuclease protein can be Cas9 or Cpf1.

In some aspects, the CRISPR-Gold system includes ribonucleoproteins. In an aspect, the CRISPR-Gold system includes Cas or Cpf1 proteins. In some aspects, the CRISPR-Gold system includes Cas9 or Cpf1/gRNA ribonucleoprotein complexes. The Cas9 or Cpf1 ribonucleoproteins can be purified Cas9 or Cpf1 proteins in complex with a gRNA. The Cas9 or Cpf1 proteins can be assembled in vitro and then subsequently delivered as a CRISPR-Gold system directly to cells using standard electroporation or transfection techniques. In an aspect, the Cas9 or Cpf1 ribonucleoproteins can cleave genomic targets. In an aspect, the CRISPR-Gold system described herein can generate single- or multi-gene knockouts via gene editing using homology directed repair.

The CRISPR-Gold system described herein, using Cas9 ribonucleoproteins or Cpf1 ribonucleoproteins can deliver intact complexes that do not require the use of cellular transcription/translation machinery to generate functional Cas9-gRNA or Cpf1-gRNA complexes.

In some aspects, Cas9 or Cpf1 or a variant thereof can be purified from bacteria. While Cas9 can require two RNA molecules to cut DNA, Cpf1 needs one RNA molecule. Cas9 and Cpf1 proteins cut DNA at different locations. For example, Cas9 can cut both strands in a DNA molecule at the same position, leaving behind blunt ends. Cpf1 can cut DNA that is staggered such that it leaves one DNA strand longer than the other DNA strand, creating sticky ends. The sticky ends can aid in the incorporation of new sequences of DNA, making Cpf1, in some instances, more efficient at gene introductions than Cas9. Cas9 and Cpf1 recognize different PAMs. In an aspect, Cas9 can use a G-rich PAM on the 3′ side. In an aspect, Cpf1 can use a T-rich PAM on the 5′ side of the guide.

In an aspect, the biodegradable polymer can be PAsp(DET). Examples of biodegradable polymers that can be used in the CRISPR-gold systems described herein include but are not limited to polyethylene imine, poly(arginine), poly(lysine), poly(histidine), poly-[2-{(2-aminoethyl)amino}-ethyl-aspartamide] (pAsp(DET)), a block co-polymer of poly(ethylene glycol) (PEG) and poly(arginine), a block co-polymer of PEG and poly(lysine).

The CRISPR-Gold system disclosed herein can also include detectable labels. Such detectable labels can include a tag sequence designed for detection (e.g., purification or localization) of an expressed polypeptide. Tag sequences include, for example, green fluorescent protein, glutathione S-transferase, polyhistidine, c-myc, hemagglutinin, or Flag™ tag, and can be fused with the oligonucleotide DNA.

In some aspects, the CRISPR-Gold system disclosed herein is capable of driving expression of one or more sequences in mammalian cells.

In some aspects, the CRISPR-Gold systems described herein can be, more generally referred to as CRISPR-carrying nanoparticles. As described herein, nanoparticles can be used to carry the CRISPR components. In some aspects, the gold nanoparticle described herein can be replaced with a silver nanoparticle. Thus, the “CRISPR-Gold system” can be a “CRISPR-silver system.” For example, in an aspect, a plurality of DNA oligonucleotides can be conjugated to a silver particle forming a DNA oligonucleotide-silver particle. In an aspect, CRISPR-silver systems can comprise: a) a plurality of DNA oligonucleotides conjugated to a silver nanoparticle forming a DNA oligonucleotide-silver nanoparticle; b) one or more RNA-guided endonuclease proteins conjugated to one or more guide RNA molecules (gRNA) forming one or more ribonucleoprotein (RNPs); and c) a biodegradable polymer.

CRISPRs are a family of DNA loci that are generally specific to a particular species (e.g., bacterial species). The CRISPR locus comprises a distinct class of interspersed short sequence repeats (SSRs) that were identified in E. coli, and associated genes. The repeats can be short and occur in clusters that are regularly spaced by unique intervening sequences with a constant length.

As used herein, the term “target sequence” refers to a sequence to which a guide sequence (e.g. gRNA) is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence can comprise any polynucleotide, such as DNA or RNA polynucleotides. In some aspects, a target sequence can be located in the nucleus or cytoplasm of a cell. In some aspects, the target sequence can be within an organelle of a eukaryotic cell (e.g., mitochondrion). A sequence or template that can be used for recombination into the targeted locus comprising the target sequences is referred to as an “editing template” or “editing polynucleotide” or “editing sequence.” Disclosed herein are target sequences. In an aspect, the target sequence(s) can be is selected from one or more of the sequences listed in Table 1.

A guide sequence (e.g. gRNA) can be any polynucleotide sequence having sufficient complementarity with a target polynucleotide sequence to hybridize with the target sequence and direct sequence-specific binding of a CRISPR-Gold system or CRISPR complex to the target sequence. In some aspects, the degree of complementarity between a guide sequence (e.g. gRNA) and its corresponding target sequence is about or more than about 50%, 60%, 70%, 75%, 80%, 85%, 90%, 95%, 99%, or more. In some aspects, a guide sequence is about more than about 5, 10, 15, 20, 25, 30, 35, 40, 45, 50 or more nucleotides in length or any number in between.

The target polynucleotide of a CRISPR complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). It is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence). A skilled person will be able to identify further PAM sequences for use with a given CRISPR enzyme. In an aspect, the PAM comprises NGG (where N is any nucleotide, (G)uanine, (G)uanine). In an aspect, the PAM comprises TTTN ((T)hymine, (T)hymine, (T)hymine and N is any nucleotide. The target sequence corresponds to one or more genes. In an aspect, the target sequence can be fragile X mental retardation 1 gene (e.g., FMR1), glutamate metabotropic receptor 5 gene (e.g., Grm5), and YFP gene (e.g., yfp). In an aspect, the target sequence can be selected from one or more of the sequences listed in Table 1. In an aspect, the target sequence of a DNA locus in a cell can be fragile X mental retardation 1 (FMRI) gene or metabotropic glutamate receptor 5 (Grm5) gene.

Disclosed herein, are gRNA sequences. The disclosed gRNA sequences can be specific for one or more desired target sequences. In some aspects, the gRNA sequence hybridizes with a target sequence of a DNA molecule or locus in a cell. In an aspect, the gRNA sequence hybridizes to one or more target or targets sequences corresponding to including but not limited to neurons and non-neuronal cells (e.g., glial cells), such as astrocytes and microglia. In some aspects, the cell can be a eukaryotic cell. In some aspects, the target sequences can be selected from one or more of the sequences listed in Table 1. In some aspects, the cell can be a mammalian or human cell. In some aspects, the cell can be located in the striatum. In some aspects, the cell can be located in the hippocampus. In some aspects, the cell can be a neuronal cell or a glial cell. In an aspect, the glial cell can be an astrocyte or a microglial cell. In some aspects, the cell can be any cell that can be delivered therapeutically to the brain, including but not limited to stem cells. For direct gene therapy and delivery to the brain, the cell type can be any cell type in the brain, including but not limited to neurons and glial cells. In an aspect, the cell in the brain can be a striatal neuron. In an aspect, gRNA sequences target one or more cell type in the brain.

In some aspects, the gRNA can target and hybridize with the target sequence and can direct the RNA-directed nuclease to the DNA locus. In some aspects, the CRISPR-Gold system disclosed herein comprises one or more gRNA sequences. In some aspects, the gRNA sequences are listed in Table 4. In some aspects, the target sequences can be selected from one or more of the sequences listed in Tables 1 and 3. In some aspects, the CRISPR-Gold system disclosed herein comprises 1, 2, 3, 4 or more gRNA sequences. In some aspects, theCRISPR-Gold system described herein comprises 1, 2, 3, 4 or more gRNA sequences in a single system. In some aspects, the gRNA sequences disclosed herein can be used turn one or more genes on or off.

In some aspects, any of the components of the CRISPR-gold system can be genetically or chemically modified. In some aspects, Cas9, Cpf1 and CRISPR variants can be genetically or chemically modified. For example, various approaches have been reported to increase the specificity of CRISPR-Cas9 to minimize off-target events. Examples of genetic or chemical modifications can include truncations and extensions at the 5′ ends of gRNAs, co-localization of paired nickase mutants of Cas9, fusion of catalytically inactive dCas9 to dimerization-dependent Fokl nuclease, and engineered higher-fidelity versions of Cas9 protein. Other approaches can include controlling the duration of CRISPR activity in eukaryotic cells, for example by transient delivery of Cas9 and gRNA as a ribonucleoprotein complex (gRNP) via cationic lipids or electroporation, by inducible temporal control, or by timed addition of a CRISPR-Cas9 inhibitor and other strategies.

In some aspects, one or more elements of a CRISPR-Gold system can be derived from a type I, type II, or type III CRISPR system. In some aspects, one or more elements of a CRISPR-Gold system can be derived from a particular organism comprising an endogenous CRISPR system, such as Streptococcus pyogenes. Generally, a CRISPR-Gold system can be characterized by elements that promote the formation of a RNA-directed endonuclease-guide RNA complex at the site of a target sequence (also referred to as a proto spacer in the context of an endogenous CRISPR system).

The compositions described herein can include a sequence corresponding to a RNA-directed nuclease. The RNA-directed nuclease can be a CRISPR-associated endonuclease. In some aspects, the RNA-directed nuclease can be a Cas9 nuclease or protein. In some aspects, the Cas9 nuclease or protein can have a sequence identical to the wild-type Streptococcus pyrogenes sequence. In some aspects, the Cas9 nuclease or protein can be a sequence for other species including, for example, other Streptococcus species, such as thermophilus; Psuedomona aeruginosa, Escherichia coli, or other sequenced bacteria genomes and archaea, or other prokaryotic microogranisms. In some aspects, the wild-type Streptococcus pyrogenes sequence can be modified. In some aspects, the nucleic acid sequence can be codon optimized for efficient expression in eukaryotic cells.

In some aspects, the RNA-directed nuclease can be a Cpf1 nuclease or protein. In some aspects, the Cpf1 nuclease or protein can have a sequence from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus. In some aspects, the Cpf1 nuclease or protein can have a sequence from an organism from S. mutans, S. agalactiae, S. equisimilis, S. sanguinis, S. pneumonia; C. jejuni, C. coli; N salsuginis, N. tergarcus; S. auricularis, S. carnosus; N. meningitides, N gonorrhoeae; L. monocytogenes, L. ivanovii; C. botulinum, C. difficile, C. tetani, C. sordellii.

In some aspects, the Cpf1 nuclease or protein can be derived from a bacterial species selected from Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae.

In some aspects, CRISPRi (CRISPR interference) can be integrated into the compositions disclosed herein. CRISPRi utilizes a nuclease-dead version of Cas9 (dCas9). In some aspects, the dCas9 can be used to repress expression of one or more target sequences (e.g., FMR1, yfp and Grm5). In some aspects, the target sequences can be selected from one or more of the sequences listed in Table 1. Instead of inducing cleavage, dCas9 remains bound tightly to the DNA sequence, and when targeted inside an actively transcribed gene, inhibition can lead to efficient transcriptional repression.

In some aspects, the CRISPR-Gold system described herein can be used to upregulate or downregulate one or more genes in the same cell. In some aspects, the CRISPR-Gold system described herein can also be used to upregulate and downregulate more than one gene or a combination thereof in the same cell. In an aspect, the expression of one or more genes (or gene products) can be decreased. In some aspects, the expression of one or more genes (or gene products) can be increased. In some aspects, the expression of one or more genes (or gene products) is increased and decreased.

Multiple gRNAs can be used to control multiple different genes simultaneously (multiplexing gene targeting), as well as to enhance the efficiency of regulating the same gene target. In some aspects, enhancer sequences can be included. Enhancer sequences are short DNA sequences (about 50 to 1,500 bp) that can be bound by proteins (e.g., activators or transcription factors) to increase the likelihood that transcription of a particular gene will occur. Enhancers can be located upstream of a gene, downstream of a gene, or within the coding region of the gene.

Disclosed herein is a CRISPR-Gold system for targeted genomic modification of Grm5 in mammalian cells. In an aspect, the system can comprise a guide RNA sequence that can target and hybridize with a sequence that can encode Grm5. In an aspect, the mammalian cells can be human cells.

Disclosed herein are guide RNA (gRNA) molecules that target one or more nucleotides in a Grm5 molecule. In an aspect, the RNA molecule can target a nucleic acid sequence that encodes the Grm5 molecule. In an aspect, the nucleic acid sequence that can encode the Grm5 molecule can comprise one or more of: a sequence encoding an amino acid sequence of the Grm5 molecule, a sequence encoding the amino acid sequence of the Grm5 molecule comprising non-translated sequence, or a sequence encoding the amino acid sequence of the Grm5 molecule comprising non-transcribed sequence. In an aspect, the nucleic acid that can encode the Grm5 molecule corresponds to SEQ ID NO: 36. In an aspect, the gRNA molecule can be configured to provide a Cas9 molecule-mediated cleavage event in the nucleic acid that can encode the Grm5 molecule. In an aspect, the gRNA molecule can target the sequence encoding an amino acid sequence of the Grm5 molecule; can be configured to provide a Cas9 molecule-mediated cleavage event in the sequence encoding an amino acid sequence of the Grm5 molecule; or can comprise a targeting domain configured to provide a Cas9 molecule-mediated cleavage event in the sequence encoding an amino acid sequence of the Grm5 molecule.

Methods

Methods of designing gRNAs. In some aspects, a commercially available tool, such as the UCSC genome browser (GRCh37/hg19), can be used to select sequences for the 5-UTR and the promoter region, 1000 base pairs upstream that can be entered into the CRISPR design tool (crispr.mit.edu). The design tool outputs 20 base pair gRNAs that are followed on their 3′ end by the PAM sequence NGG, which is specific to the CRISPR-Cas9 system derived from Streptococcus pyogenes. The design tool can also score the potential gRNA sequences based on the number of off-target sties they may have and how many are within genes. The score ranges from 0-100, with a higher score meaning less off-target sites within genes. Guide RNAs described herein, for example, that had a score of 75 and above were selected for further study. The selected gRNAs can then be entered into the BLAT tool of the UCSC genome browser to inspect for overlap of gRNAs with DNAse hypersensitivity sites to ensure overlap. Any site that has DNAse hypersensitivity value above 0.01 can be targeted with a guide if one is available from the list of guides generated as described above. Additionally, any site that shows greater than 10 transcription factor binding sites within a region, as determined from ChiP-seq, can also be considered. Generally, the DNAse hypersensitivity data is consistent with these regions. Using the criteria described above, gRNAs (e.g., 4-7 gRNAs) that are spaced at least 100 base pairs apart can be selected for performing targeted gene repression and screening. In an aspect, FMR1 and mGlur5 gRNAs guides can be screened using the method disclosed herein. In an aspect, gRNA sequences from the promoter region and 5′UTR (crispr.mit.edu) can be selected. In an aspect, gRNA sequences are 20 bp in length followed by a PAM sequence (e.g., 3′ NGG, 5′TTN, 5′ TTTN). In an aspect, gRNA sequences with the least off-target sequences and those that overlap with DNase sensitivity peaks can be selected.

Disclosed herein are methods of modulating expression of a gene in a cell. In an aspect, the method can include introducing into a cell a CRISPR-Gold system as disclosed herein. In an aspect, the CRISPR-Gold system, can comprise: a plurality of DNA oligonucleotides conjugated to a gold nanoparticle forming a DNA oligonucleotide-gold nanoparticle (GNP); one or more RNA-guided endonuclease proteins conjugated to one or more guide RNA molecules (gRNA) forming one or more ribonucleoprotein (RNPs), wherein the gRNA is complementary to a target nucleic acid sequence comprising the gene; and a biodegradable polymer. In an aspect, the one or more RNPs can be conjugated to the GNP forming a RNP-GNP complex, and wherein the biodegradable polymer can encapsulate the RNP-GNP complex thereby forming the CRISPR-Gold system. In an aspect, the cell can produce the gRNA and the gRNA can hybridize with a target sequence of a DNA locus in a cell. In an aspect, the gRNA can target and hybridize with a target sequence and can direct the one or more RNA-guided endonuclease proteins to the DNA locus. In an asepct, the DNA locus can modulate the expression of the gene. In an aspect, the cell and the gRNA sequence can be selected from Table 4.

Disclosed herein, are methods for introducing into a cell a CRISPR-Gold system. In an aspect, the method can include introducing into a cell a CRISPR-Gold system as disclosed herein. In an aspect, the CRISPR-Gold system can comprise: a plurality of DNA oligonucleotides conjugated to a gold nanoparticle forming a DNA oligonucleotide-gold nanoparticle (GNP); one or more RNA-guided endonuclease proteins conjugated to one or more guide RNA molecules (gRNA) forming one or more ribonucleoprotein (RNPs), wherein the gRNA is complementary to a target nucleic acid sequence comprising the gene; and a biodegradable polymer. In an aspect, the one or more RNPs can be conjugated to the GNP forming a RNP-GNP complex, and wherein the biodegradable polymer can encapsulate the RNP-GNP complex thereby forming the CRISPR-Gold system. In an aspect, the cell can produce the gRNA and the gRNA can hybridize with a target sequence of a DNA locus in a cell. In an aspect, the gRNA can target and hybridize with a target sequence and can direct the one or more RNA-guided endonuclease proteins to the DNA locus. In an asepct, the DNA locus can modulate the expression of the gene. In an aspect, the cell and the gRNA sequence can be selected from Table 4.

Disclosed herein are methods for targeted genomic modification of Grm5 in mammalian cells. In an aspect, the methods can comprise administering a CRISPR-Gold nanoparticle. In an aspect, the CRISPR-Gold nanoparticle can comprise a guide RNA sequence that can target and hybridize with a sequence that can encode Grm5.

Disclosed herein, are methods for inducing site-specific DNA cleavage in a cell. The method can include contacting a cell with a guide RNA. The guide RNA can be selected from Table 4. The guide RNA can include a sequence capable of binding to a target DNA. The method can further comprise the following step: contacting the cell with a RNA-guided endonuclease protein. In an aspect, the DNA can be in a cell. In an aspect, the cell can be a eukaryotic cell. In an aspect, the cell can be in an individual. In an aspect, the individual can be a human.

The method steps described herein can be carried out simultaneously or sequentially in any order. In some aspects, the DNA can be in a cell. In some aspects, the cell can be a eukaryotic cell. In some aspects, the cell can be in an individual. In some aspects, the individual can be a human.

Method of Treatment

The methods disclosed herein can be useful for the treatment of a subject having a brain disorder or disease. The methods disclosed herein can be effective for targeting one or more genes, FMR1 and Grm5. In some aspects, the methods can also include the step of administering a therapeutic effective amount of the compositions disclosed herein (e.g., a CRISPR-Gold system). In an aspect, the CRISPR-Gold system can comprise: a plurality of DNA oligonucleotides conjugated to a gold nanoparticle forming a DNA oligonucleotide-gold nanoparticle (GNP); one or more RNA-guided endonuclease proteins conjugated to one or more guide RNA molecules (gRNA) forming one or more ribonucleoprotein (RNPs), wherein the gRNA is complementary to a target nucleic acid sequence comprising the gene; and a biodegradable polymer. In an asepct, the gRNA sequence is selected from the group listed in Table 4. In some aspects, the target sequences can be selected from one or more of the sequences listed in Table 1.

Disclosed herein are methods of treating a subject having fragile X syndrome. In an asepct, the method can comprise (a) determining mGluR 5 signaling or a mGluR5-mediated behavioral phenotype in the subject; and (b) administering to the subject a pharmaceutical composition comprising a CRISPR-Gold system comprising one or more RNA-guided endonuclease proteins conjugated to one or more guide RNA molecules (gRNA), wherein the gRNA is SEQ ID NO: 36. In some aspects, the method of treating a subject having fragile X syndrome can comprise administering to the subject a therapeutically effective amount of the CRISPR-Gold system or any of the pharmaceutical compositions disclosed herein. In an aspect, the gRNA can be SEQ ID NO: 36. In an aspect, the method can further comprise identifying a subject having fragile X syndrome. In an aspect, the fragile X syndrome can be caused by increased metabotropic glutamate receptor 5 (mGluR5) signaling. In an aspect, the composition can be administered into the brain or striatum.

Disclosed herein are methods of treating a subject, the method comprising contacting a cell or a subject with an effective amount of a gRNA molecule as disclosed herein. In an asepct, the method can further comprise altering the sequence of the target nucleic acid. In an aspect, the the cell can be a vertebrate, mammalian or human cell. In an aspect, the cell can be a brain cell.

In some aspects, the methods can further include the step of identifying a subject (e.g., a human patient) who has a brain disease or disorder (e.g., fragile X syndrome) and then providing to the subject a composition comprising the CRISPR-Gold system disclosed herein. In some aspects, the brain disease or disorder can be fragile X syndrome. In some aspects, the brain disease or disorder can be caused by exgaggerated (or increased) mGluR5 signaling. In some aspects, the subject can be identified using standard clinical tests known to those skilled in the art. An example of a tests for diagnosing fragile X syndrome incude genetic testing (e.g., to identify a triplet repeat in the FMR1 gene). Subjects can also be identified as having signs or symptoms of fragile X syndrome that include but are not limited to intellectual disabilities, ranging from mild to severe; attention deficit and hyperactivity, anxiety and unstable mood, autistic behaviors (e.g., hand flapping and not making eye contact), sensory integration problems (e.g, hypersensitivity to loud noises or bright lights), speech delay, seizures. Other physical signs include but are not limited to long face, large prominent ears, flat feet, hyperextensible joints, and low muscle tone.

The therapeutically effective amount can be the amount of the composition administered to a subject that leads to a full resolution of the symptoms of the condition, disease or disorder, a reduction in the severity of the symptoms of the condition, disease or disorder, or a slowing of the progression of symptoms of the condition, disease or disorder. The methods described herein can also include a monitoring step to optimize dosing. The compositions described herein can be administered as a preventive treatment or to delay or slow the progression of of the condition, disease or disorder.

The compositions disclosed herein can be used in a variety of ways. For instance, the compositions disclosed herein can be used for direct delivery of modified therapeutic cells. The compositions disclosed herein can be used or delivered or administered at any time during the treatment process. The compositions described herein including cells can be delivered to intracranially to the brain region affected (e.g., the striatum).

In some aspects, the compositions disclosed herein can be administered or delivered to neurons (e.g., striatal neurons).

The dosage to be administered depends on many factors including, for example, the route of administration, the formulation, the severity of the patient's condition/disease/pain, previous treatments, the patient's size, weight, surface area, age, and gender, other drugs being administered, and the overall general health of the patient including the presence or absence of other diseases, disorders or illnesses. Dosage levels can be adjusted using standard empirical methods for optimization known by one skilled in the art. Administrations of the compositions described herein can be single or multiple (e.g., 2- or 3-, 4-, 6-, 8-, 10-, 20-, 50-, 100-, 150-, or more fold). Further, encapsulation of the compositions in a suitable delivery vehicle (e.g., polymeric microparticles or implantable devices) can improve the efficiency of delivery. In some aspects, the CRIRPR-gold systems described herein can be delivered with other polymers, with other types of encapsulations or without any encapsulation. Examples of other polymers include but are not limited to polyethylene imine, poly(arginine), poly(lysine), poly(histidine), poly-[2-{(2-aminoethyl)amino}-ethyl-aspartarnide] (pAsp(DET)), a block co-polymer of poly(ethylene glycol) (PEG) and poly(arginine), a block co-polymer of PEG and poly(lysine).

The therapeutically effective amount of the compositions described herein can include a single treatment or a series of treatments (i.e., multiple treatments or administered multiple times). Treatment duration using any of compositions disclosed herein can be any length of time, such as, for example, one day to as long as the life span of the subject (e.g., many years). For instance, the composition can be administered daily, weekly, monthly, yearly for a period of 5 years, ten years, or longer. The frequency of treatment can vary. For example, the compositions described herein can be administered once (or twice, three times, etc.) daily, weekly, monthly, or yearly for a 15 period of 5 years, ten years, or longer.

In some aspects, the compositions disclosed herein can also be co-administered with another therapeutic agent. The therapeutic agent can be any drug that is currently available. For example, therapeutic agents that can be co-admininistered with any of the compositions disclosed herein include, but are not limited to, peptides, hormones, neurotransmitters, neurstimulants, small molecules, antibodies, etc. In some aspects, the therapeutic agent can be sedatives, muscle relaxants, antipsychotics, cognition-enhancing medications, and vitamins.

In some aspects, the methods disclosed herein can also include treating a subject having fragile X syndrome. In some aspects, the method disclosed herein can also include treating a subject having increased or exaggereated mGlurR5 signaling. In an aspect, the increased or exaggereated mGlurR5 signaling can be in the striatal circuit, or associated with striatal neurons. In some aspects, the methods disclosed herein can include the step of determining mGluR 5 signaling or a mGluR5-mediated behavioral phenotype in a subject. In some aspects, the disclosed methods can further include the step of administering to the subject a pharmaceutical composition comprising the CRISPR-Gold system disclosed herein. In an aspect, the CRISPR-Gold system can comprise: a plurality of DNA oligonucleotides conjugated to a gold nanoparticle forming a DNA oligonucleotide-gold nanoparticle (GNP); one or more RNA-guided endonuclease proteins conjugated to one or more guide RNA molecules (gRNA) forming one or more ribonucleoprotein (RNPs), wherein the gRNA is complementary to a target nucleic acid sequence comprising the gene; and a biodegradable polymer. In an aspect, the guide RNA can be selected from the group listed in Table 4. In some aspects, the CRISPR-associated endonuclease can optimized for expression in a human cell.

Pharmaceutical Compositions

As disclosed herein, are pharmaceutical compositions, comprising the compositions disclosed herein. For example, disclosed are pharmaceutical compositions comprising the CRISPR-gold system. In an aspect, the CRISPR-Gold system can comprise: a plurality of DNA oligonucleotides conjugated to a gold nanoparticle forming a DNA oligonucleotide-gold nanoparticle (GNP); one or more RNA-guided endonuclease proteins conjugated to one or more guide RNA molecules (gRNA) forming one or more ribonucleoprotein (RNPs), wherein the gRNA is complementary to a target nucleic acid sequence comprising the gene; and a biodegradable polymer. In an aspect, the guide RNA can be selected from the group listed in Table 4. In some aspects, the target sequence can be selected from one or more of the sequences listed in Table 1. In some aspects, the pharmaceutical compositions comprise the any one of the CRISPR-Gold system disclosed herein. In some aspects, the pharmaceutical compositions can further comprise a pharmaceutically acceptable carrier.

Disclosed herein are pharmaceutical compositions comprising a guide RNA molecule as described herein.

As used herein, the term “pharmaceutically acceptable carrier” refers to solvents, dispersion media, coatings, antibacterial, isotonic and absorption delaying agents, buffers, excipients, binders, lubricants, gels, surfactants that can be used as media for a pharmaceutically acceptable substance. The pharmaceutically acceptable carriers can be lipid-based or a polymer-based colloid. Examples of colloids include liposomes, hydrogels, microparticles, nanoparticles and micelles. The compositions can be formulated for administration by any of a variety of routes of administration, and can include one or more physiologically acceptable excipients, which can vary depending on the route of administration. Any of the components of the CRISPR-Gold system, for example, the gRNAs described herein can be administered in the form of a pharmaceutical composition.

As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed. The compositions can also include additional agents (e.g., preservatives).

The pharmaceutical compositions as disclosed herein can be prepared for oral or parenteral administration. Pharmaceutical compositions prepared for parenteral administration include those prepared for intravenous (or intra-arterial), intramuscular, intervertebral subcutaneous, facet joint, dorsal root ganglion, intrathecal or intraperitoneal administration. Paternal administration can be in the form of a single bolus dose, or may be, for example, by a continuous pump. In some aspects, the compositions can be prepared for parenteral administration that includes dissolving or suspending the CRISPR-Gold systems, nucleic acids or polypeptide sequences in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

In some aspects, the CRISPR-Gold system disclosed herein can be directly injected into the brain without a carrier or with a biomaterial carrier. Any hydrogel or biomaterial designed for non-viral delivery can be used. In some aspects, the cells can be administered to a desired location with or without a biomaterial carrier.

In some aspects, the compositions disclosed herein or the CRISPR-Gold system disclosed herein can be administered with or without a carrier to one or more neurons. In some aspects, the one or more neurons can be in the striatum. Thus, the administration of the compositions or CRISPR-Gold system disclosed herein can be delivered (e.g., injected) locally (e.g., to the neuron site).

In some aspects, the compositions disclosed herein are formulated for systemic or intracranial administration.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment. The compositions can also be formulated as powders, elixirs, suspensions, emulsions, solutions, syrups, aerosols, lotions, creams, ointments, gels, suppositories, sterile injectable solutions and sterile packaged powders. The active ingredient can be nucleic acids or polypeptides described herein in combination with one or more pharmaceutically acceptable carriers. As used herein “pharmaceutically acceptable” means molecules and compositions that do not produce or lead to an untoward reaction (i.e., adverse, negative or allergic reaction) when administered to a subject as intended (i.e., as appropriate).

In some aspects, the CRISPR-Gold system, gRNAs and nucleic acid sequences as disclosed herein can be delivered to a cell of the subject. In some aspects, such action can be achieved, for example, by using polymeric, biodegradable microparticle or microcapsule delivery vehicle, sized to optimize phagocytosis by phagocytic cells (e.g., macrophages). In some aspects, the CRISPR-Gold system described herein can include an additional coating.

In some aspects, the formulations include any that are suitable for the delivery of a CRISPR-Gold systems and cells. In an aspect, the route of administration includes but is not limited to injection into the brain or into the striatum.

Kits

The kits described herein can include any combination of the compositions (e.g., CRISR-Cas system) described above and suitable instructions (e.g., written and/or provided as audio-, visual-, or audiovisual material). In an aspect, the kit comprises a predetermined amount of a composition comprising any one of the CRISPR-Gold systems or compositions disclosed herein. The kit can further comprise one or more of the following: instructions, sterile fluid, syringes, a sterile container, delivery devices, and buffers or other control reagents.

### EXAMPLES

### Example 1: CRISPR-Gold Delivers Cas9 and Cpf1 Ribonucleoproteins to an Adult Mouse Brain and Induces Deletion of Target Genes in Astrocytes, Microglia and Neurons

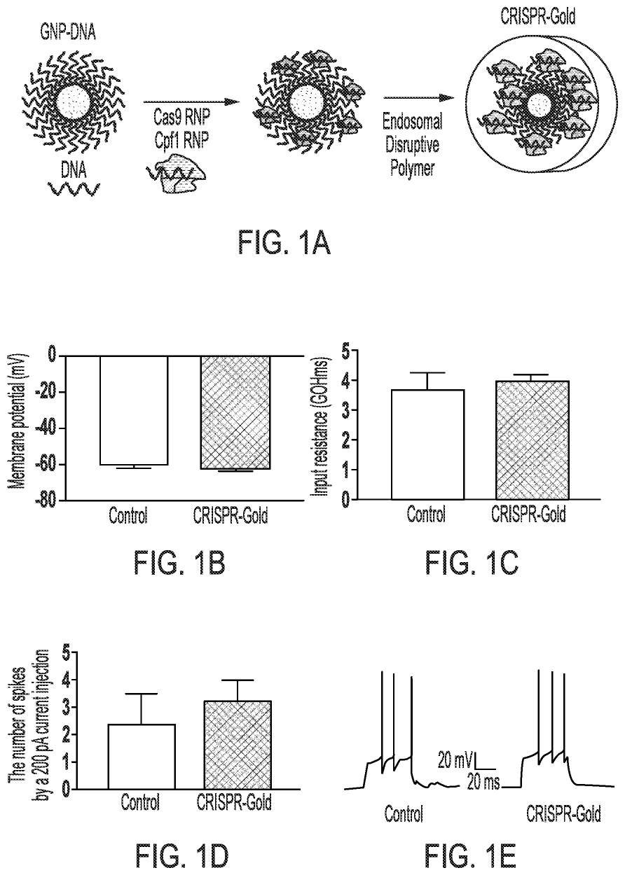

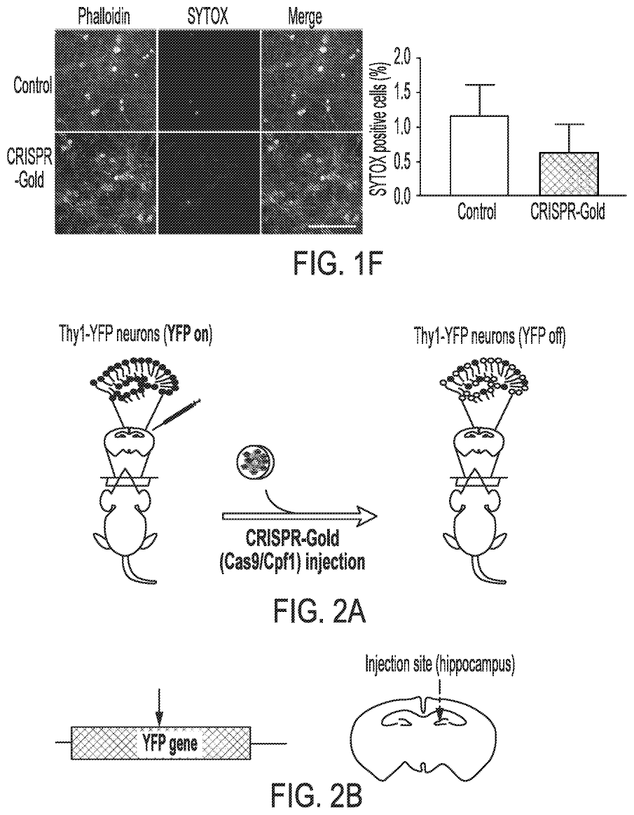

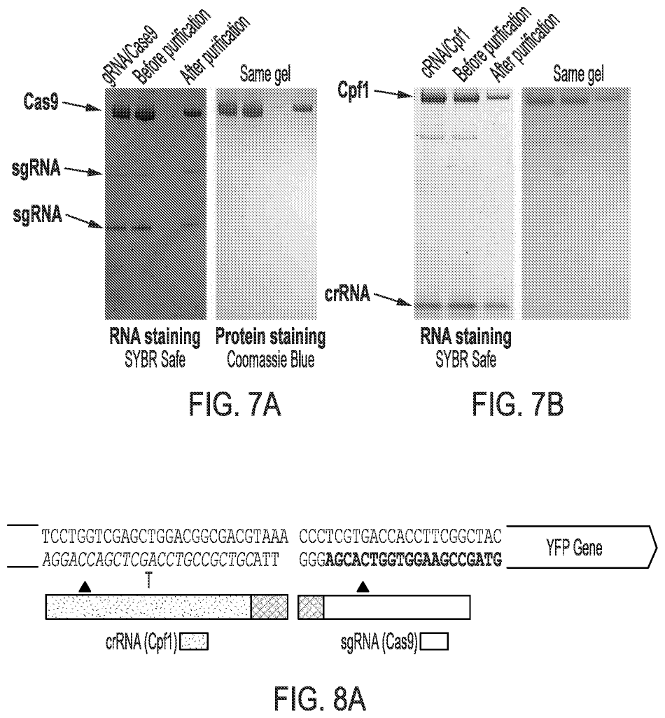

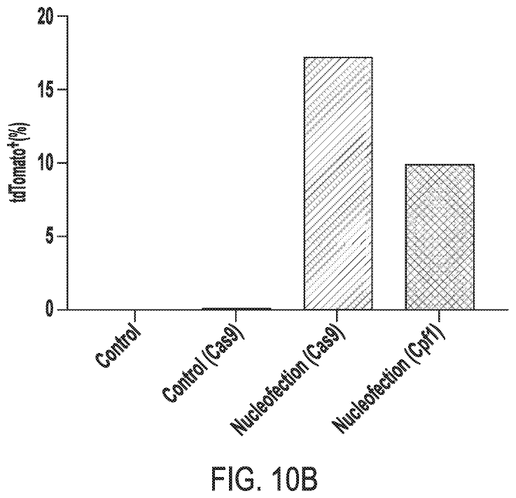

Non-viral delivery vehicles that can edit genes in the brain have potential for treating neurological disorders. However, little is known about non-viral gene editing in the brain. CRISPR-Gold (FIG. 1a) was identified as a delivery vector for gene editing in the brain, because it was able to deliver Cas9 RNPs into a variety of cell types in vitro and into mouse muscles efficiently (Lee, K. et al. Nat Biomed Eng 1, 889-901 (2017)). DNA oligonucleotide-conjugated gold nanoparticles (GNPs) bind to Cas9 or Cpf1 RNPs and PAsp(DET) polymer encapsulation which generates CRISPR-Gold. In order to investigate the biocompatibility of CRISPR-Gold in neuronal cells, the cytotoxicity and physiological effects of primary cultured hippocampal neurons treated with CRISPR-Gold was measured. The electrophysiological properties (whole-cell current clamp recording) of pyramidal neurons after treatment with CRISPR-Gold loaded with Cas9 RNPs (the CRISPR-Gold complex was verified in FIG. 7) were checked first. For this, RNP-Gold complex was loaded on a polyacrylamide gel with Tris-SDS buffer, causing dissociation of the RNPs from gold nanoparticles. sgRNA/Cas9 and crRNA/Cpf1 are controls. SYBR Safe stains nucleic acid and shows that (a) Cas9 binding to sgRNA and (b) Cpf1 binding to crRNA. The same gel is additionally stained with Coomassie Blue to visualize proteins. CRISPR-Gold after purification shows clear bands of both Cas9 RNPs and Cpf1 RNPs, which means that the RNPs are loaded to CRISPR-Gold.The membrane potentials were no different between control and treated neurons (FIG. 1b). Input resistance, which in part indicates the leakiness of the plasma membrane, was not significantly different from the untreated neurons (FIG. 1c). Consistent with a similar input resistance, the number of spikes generated by a 200 pA current injection did not significantly change in CRISPR-Gold treated neurons (summarized in FIG. 1d, representative traces in 1e). No significant difference in the membrane potentials, input resistance, and the number of spikes were found between groups. In FIG. 1F, neurons were fixed 14 days after CRISPR-Gold treatment and stained with SYTOX-Red (Red) for staining dead cells and phalloidin-Alexa 488 (Green) for visualizing neuronal morphology. Therefore, it was concluded that CRISPR-Gold treatment does not have adverse effects on neuronal membrane health, or specifically affect neuronal excitability. Next, dead cells were stained with SYTOX, and morphology was visualized by co-staining actin with a phalloidin stain to check the cytotoxicity of CRISPR-Gold treatment. No significant differences in the number of dead cells and neuronal morphology were found in CRISPR-Gold treated cells compared to untreated neurons (FIG. 10. Taken together, these results show that treatment with CRISPR-Gold is not cytotoxic nor does it affect the physiological function of neurons.

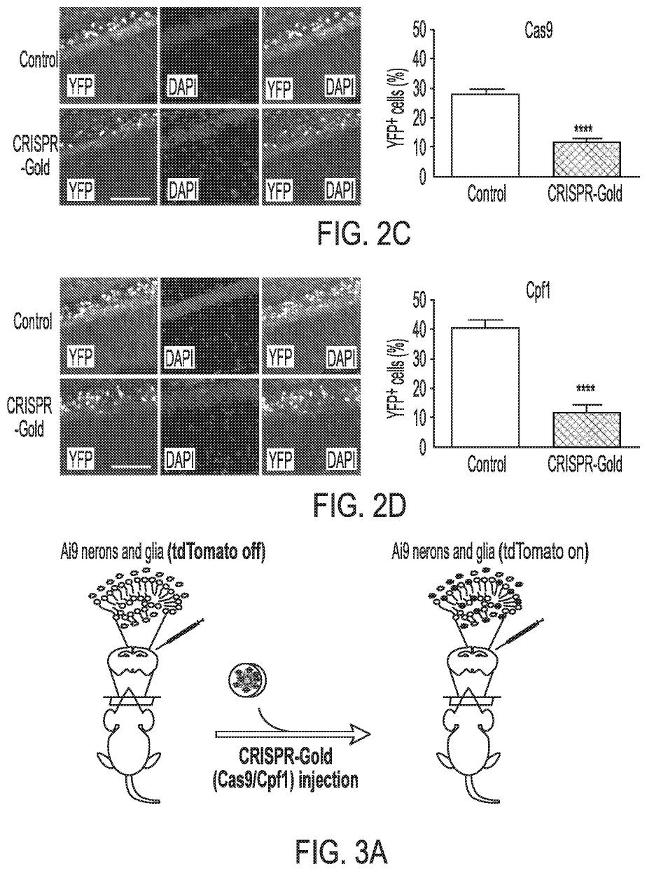

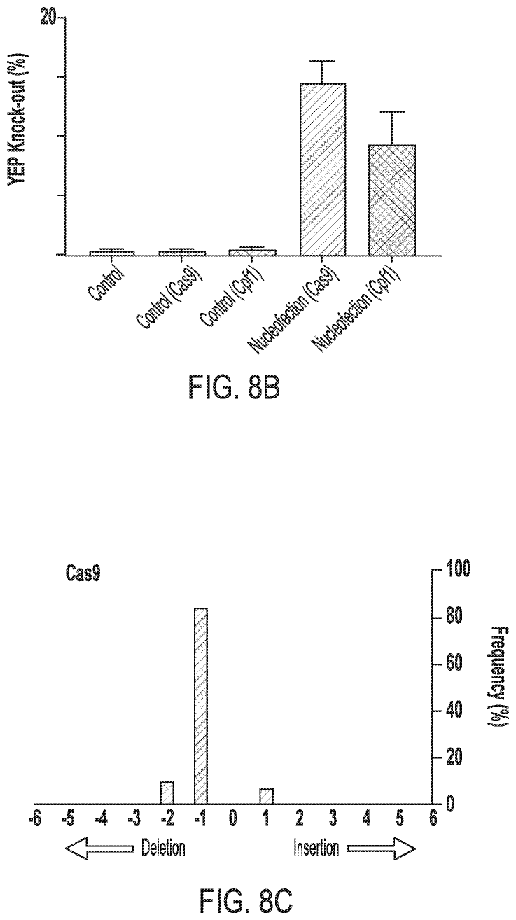

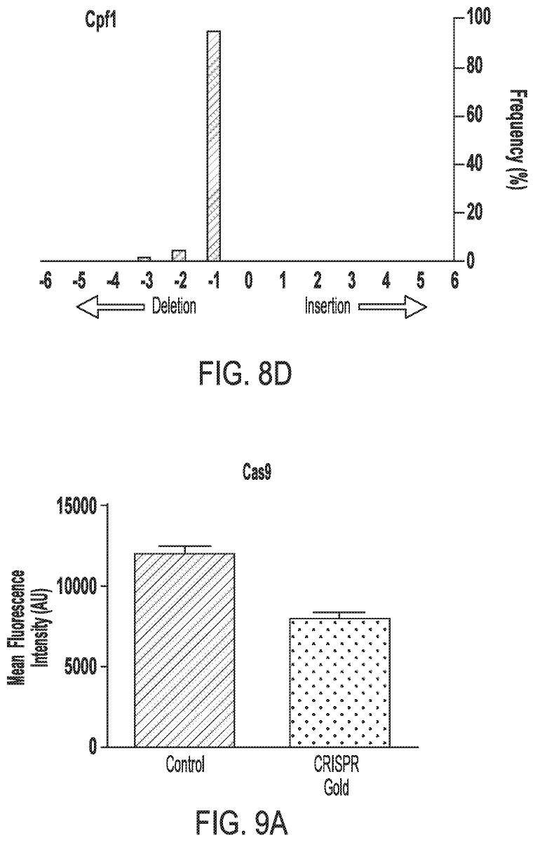

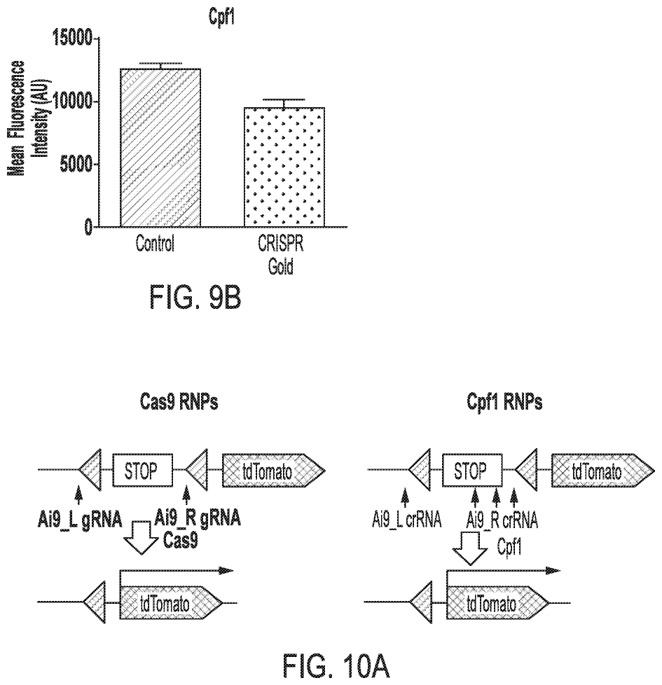

To test whether CRISPR-Gold can deliver Cas9 RNPs in an adult mouse brain, CRISPR-Gold was stereotaxically injected into the brains of adult mice (FIG. 2a). Neurons were selected as the initial target for gene editing investigations because of their function in brain activities and their correlation with a wide variety of neurological diseases. The Thy1-YFP mouse model, a transgenic mouse line that expresses YFP in neurons, but not in other types of brain cells (Feng, G. et al. Neuron 28, 41-51 (2000)), was used to monitor gene editing in neurons (FIG. 2a). The sgRNAs for Cas9 and crRNAs for Cpf1 were designed to target the 5′ region of the YFP gene to induce indel mutations (FIG. 8a), and these guide RNAs were verified by checking YFP gene knockout capability in YFP-expressing HEK cells (FIGS. 2b-2d). Indel mutations caused by Cas9 or Cpf1 induce knocking out of YFP gene expression. Control is a negative control with no treatment. Control (Cas9 or Cpf1) cells were treated with Cas9 RNPs without nucleofection. Nucleofection (Cas9 or Cpf1) cells were nucleofected with Cas9/sgRNAs or Cpf1/crRNAs. Efficient yfp knockout was observed from both Cas9 and Cpf1-nucleofected cells as a result. One or two base deletions are the most common mutations after Cas9 or Cpf1 delivery when targeting the YFP gene. CRISPR-Gold loaded with Cas9 or Cpf1 RNPs targeting the 5′ region of the YFP gene was then injected into the dentate gyrus in the hippocampus of 1-2 month-old adult mice (FIG. 2b). FIG. 2c-2d and FIG. 9 shows that CRISPR-Gold can deliver Cas9 or Cpf1 RNPs and efficiently edit the YFP gene in neurons that are projecting to the molecular layer of the dentate gyrus after the stereotaxic injection of CRISPR-Gold with Cas9 or Cpf1 RNPs. As a result, there was approximately a 17% or 28% decrease in YFP⁺ cells in the granular layer of dentate gyrus by Cas9 or Cpf1 respectively (FIG. 2c and 2d) or a 34% or 25% decrease in YFP expression levels (FIGS. 9a and 9b) in the molecular layer of the dentate gyms after the delivery of Cas9 or Cpf1, respectively. Quantification of the YFP intensity (mean fluorescence intensity) in the molecular layer of the dentate gyrus in the injected side (CRISPR-Gold) compared to the contralateral control side (Control). These results demonstrate that Cas9 and Cpf1 are efficient methods for in vivo editing in the brain by using CRISPR-Gold. Given that modulating a small subpopulation of neurons (200-300 neurons) in a particular brain region could led to the change of behavioral outputs in previous studies (Choi, G. B. et al. Cell 146, 1004-1015 (2011); and Fried, I., Mukamel, R. & Kreiman, G. Internally generated preactivation of single neurons in human medial frontal cortex predicts volition. Neuron 69, 548-562 (2011)), local treatment of CRISPR-Gold has the potential to treat a wide variety of neurological disorders and to elucidate the role of genes in local brain regions.

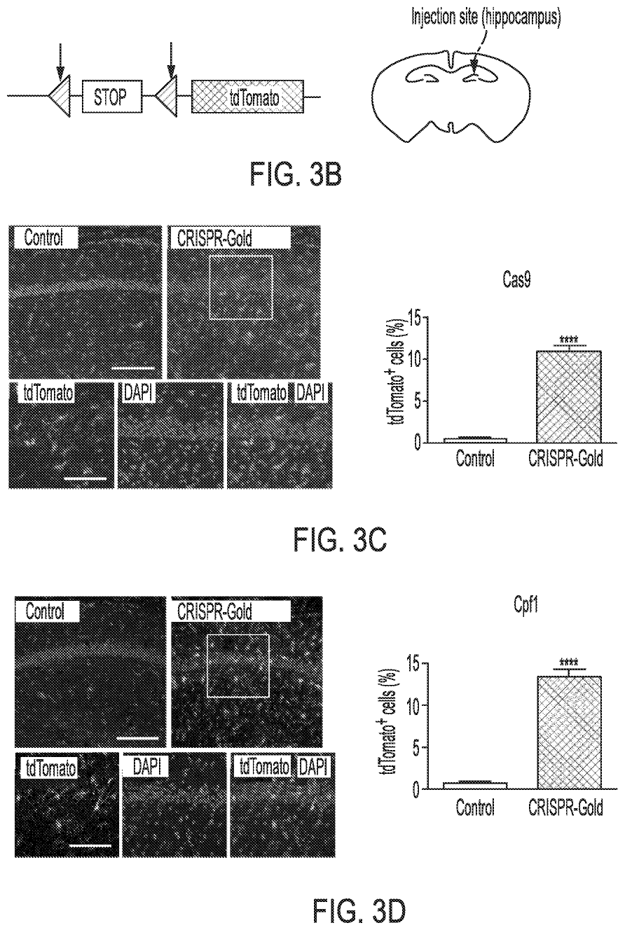

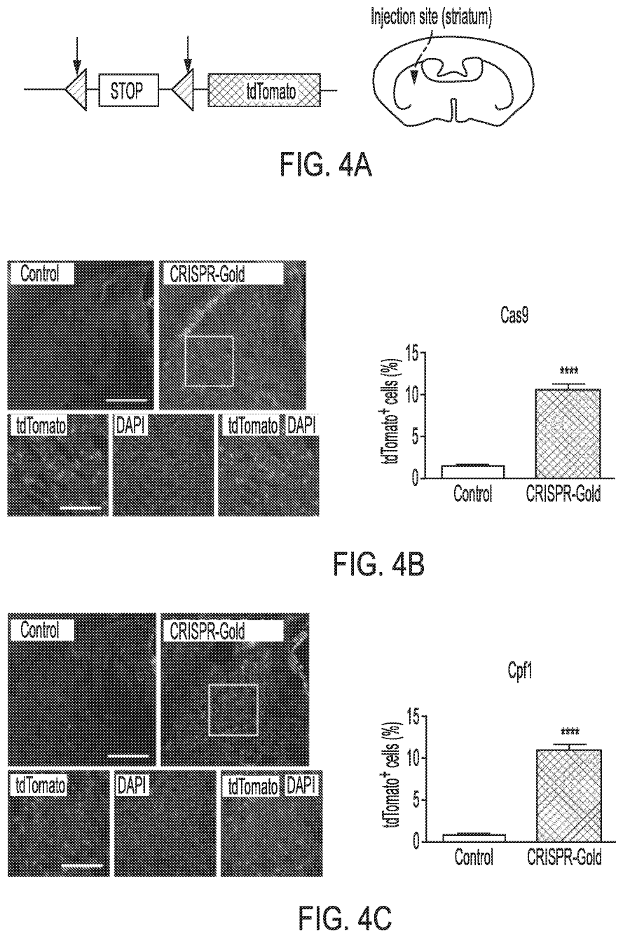

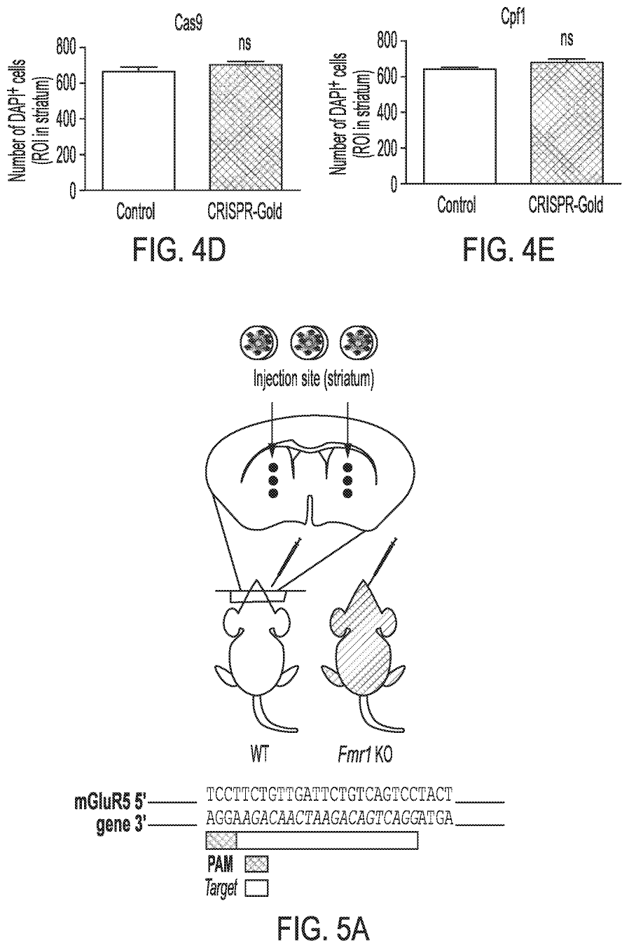

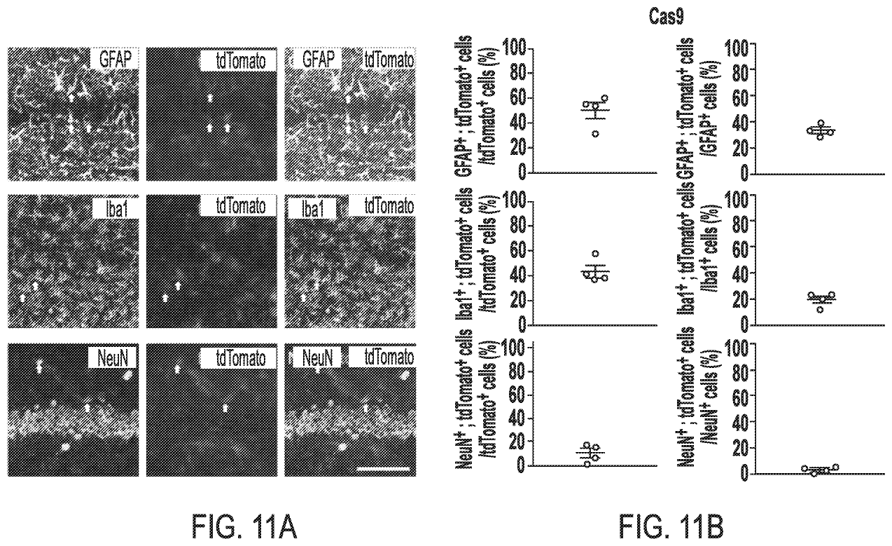

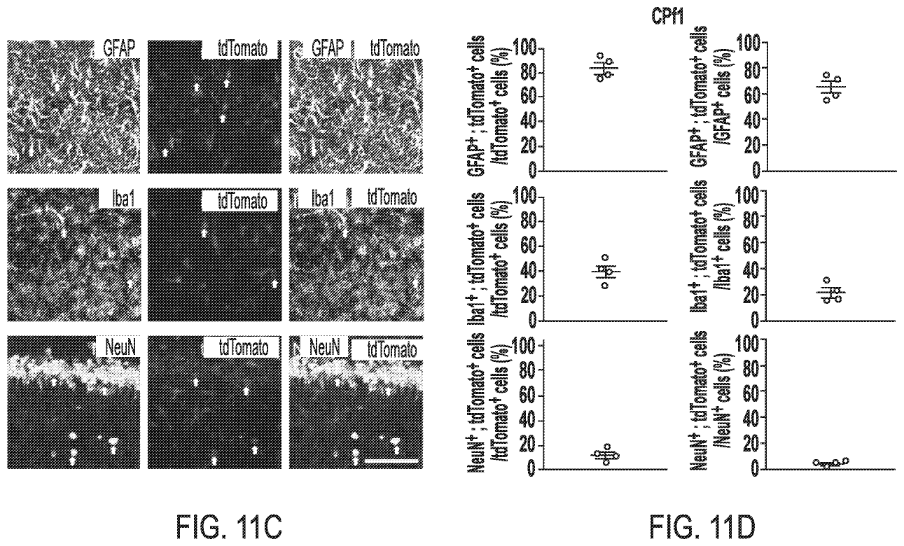

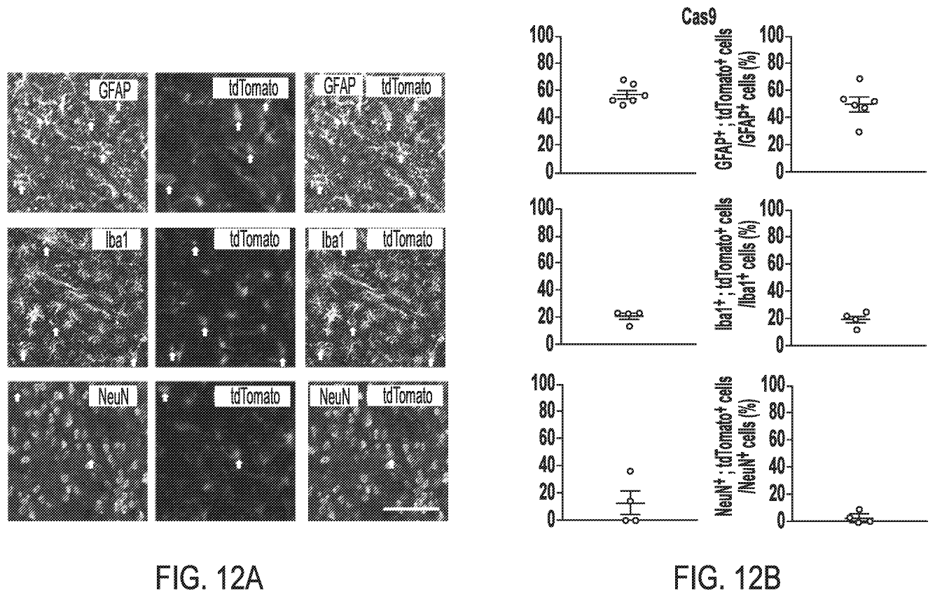

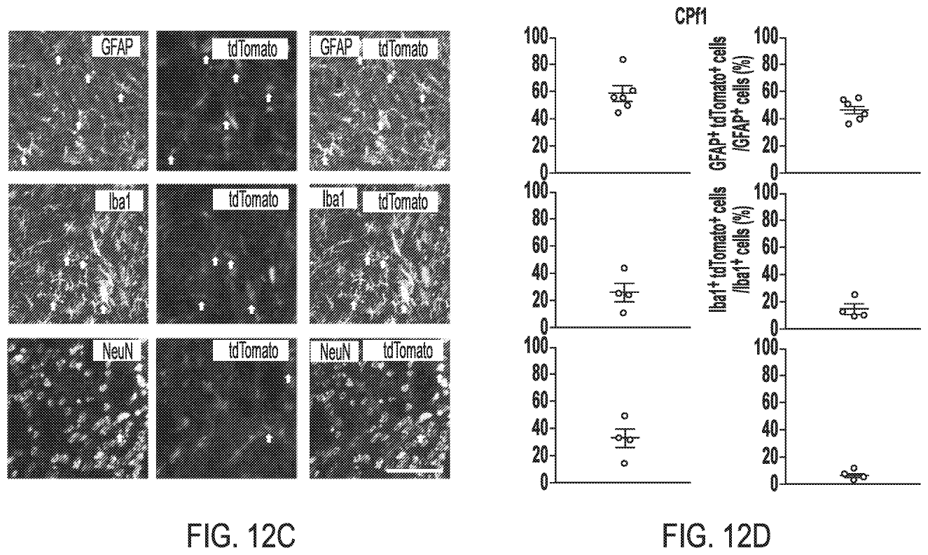

Gene editing via deletion of repeated genetic sequences in a human patient's brain can be a therapeutic treatment to cure disorders such as Huntington's disease and FXS, which have repeated sequences that result in brain dysfunctions (McMahon, M. A. & Cleveland, D. W. Nat Rev Neurol 13, 7-9 (2017); Xie, N. et al. PLoS One 11, e0165499 (2016); and Park, C. Y. et al. Cell Rep 13, 234-241 (2015)). Therefore, experiments were performed to determine if Cas9 and Cpf1 RNPs could induce genome editing by deletion of a target gene in an adult mouse brain using Ai9 mice (FIGS. 3a, 3b, and 4a). The Ai9 mouse is a genetically engineered mouse model, which has a fluorescent tdTomato gene with a stop sequence upstream of it (Madisen, L. et al. Nat Neurosci 13, 133-140 (2010)). In this mouse model, tdTomato is silent because of the stop signal, but the deletion of the stop sequences allows transcription of the tdTomato gene, resulting in fluorescence expression (FIG. 3a). sgRNAs for Cas9 and crRNAs for Cpf1 were designed to target both ends of the stop sequences to remove them, leading to expression of tdTomato (FIG. 10a). The stop sequence is located upstream of tdTomato to stop the expression of tdTomato. The gRNAs are designed to target the 5′ and 3′ ends of the stop sequence to induce deletion, which induces the expression of tdTomato. These guide RNAs were verified in primary fibroblasts cultured from Ai9 mice, and were able to induce the expression of tdTomato (FIG. 10b). The primary cultured fibroblasts were treated with Cas9 or Cpf1, and the % of tdTomato-expressing cells was measured with flow cytometry. Control is a negative control with no treatment. Control (Cas9) cells were treated with Cas9 RNPs without nucleofection. Nucleofection (Cas9 or Cpf1) cells were nucleofected with Cas9/gRNAs or Cpf1/crRNAs. Cas9 or Cpf1-loaded CRISPR-Gold was stereotaxically injected into two brain regions (the hippocampus and the striatum of 1-2 month old adult Ai9 mice as shown in FIGS. 3b and 4a) and the expression of tdTomato was measured via fluorescence histology. FIGS. 3c and 3d demonstrate that Cas9 and Cpf1 CRISPR-Gold complexes can induce deletion of their target sequences, and can induce the expression of tdTomato in the CA1 region of the hippocampus of Ai9 mice. For example, fluorescence histology images of the hippocampus area treated with Cas9 and Cpf1 CRISPR-Gold complexes showed a clear expression of tdTomato in comparison to the contralateral control side (FIGS. 3c and 3d). Approximately 10 to 15% of the cells in the injected area of the hippocampus were tdTomato positive in both Cas9 and Cpf1 RNP-injected brains, which were quantified by analyzing the percentage of tdTomato and DAPI double positive cells (FIGS. 3c and 3d). Similar results were found in the striatum (FIGS. 4b and 4c). For example, tdTomato expresses in the striatum after injecting Cas9 and Cpf1 CRISPR-Gold complexes compared to the contralateral control side (FIGS. 4b and 4c). Approximately 10% of the cells in the injected area of the striatum were tdTomato positive in both Cas9 and Cpf1 RNP-injected brains (FIGS. 4b and 4c), suggesting that Cas9 and Cpf1 RNPs can efficiently delete targeted DNA sequences using the CRISPR-Gold delivery vehicle. The gene-edited area was 1-2 mm×1-2 mm for the hippocampus or striatum from the injection sites, and within these regions, there was no observable change in cell density by counting DAPI⁺ cells (FIGS. 4d and 4e), indicating no significant adverse effect on cell viability. Two weeks after stereotaxic injection of Cas9 or Cpf1 RNPs using the CRISPR-Gold system into the striatum of Ai9 mice, the brains were sliced and immunostained with tdTomato antibodies and stained with DAPI. The number of DAPI⁺ cells of equal size in the injected region of interest (ROI) of the striatum in both the Control group and the CRISPR-Gold group were compared and analyzed.

Whereas neurons are the basic working units of the brain, non-neuronal cells are designed to play a role in maintaining, supporting, and regulating neuronal functions. Glial cell dysfunction causes multiple brain disorders (Almad, A. A. & Maragakis, N. J. Stem Cell Res Ther 3, 37 (2012)), and there is interest in editing the genes of glial cells. Therefore, the brain cell types edited by Cas9 or Cpf1 RNPs delivered by CRISPR-Gold were further identified using Ai9 mice to determine whether glial cells were edited. CRISPR-Gold injected Ai9 brain sections were stained with the following cell markers: glial fibrillary acidic protein (GFAP), ionized calcium-binding adapter molecule 1 (Iba1) and neuronal nuclear protein (NeuN), which identify the astrocyte, microglia, and neuronal populations respectively in the histology sections. FIGS. 11 and 12 demonstrate that gene-edited tdTomato-expressing cells were composed of these cell types: astrocytes, microglia, and neurons. In the hippocampus, more than a half of tdTomato⁺ cells had the astrocyte marker GFAP in Cas9 or Cpf1 RNP-injected brains, and Iba1- and NeuN-stained cells accounted for approximately 40% and 10% of tdTomato⁺ cells respectively (FIG. 11b and 11d, left panels), suggesting that astrocytes, microglia, and neurons are edited by CRISPR-Gold Cas9 and Cpf1 in the hippocampus. Similar results were found in the striatum. In the striatum, more than a half of tdTomato⁺ cells had the astrocyte marker GFAP in Cas9 or Cpf1 injected brains, and Iba1 and NeuN stained cells accounted for 10-30% of tdTomato⁺ in Cas9 or Cpf1 injected brains (FIGS. 12b and 12d, left panels). On the other hand, 33% and 65% of GFAP⁺, 19% and 21% of Iba1⁺, or 3% and 5% of NeuN⁺ cells were edited among astrocytes, microglia, or neurons with Cas9 or Cpf1 in the hippocampus (FIG. 11b and 11d, right panels). Similar results were found in the striatum: 50% and 46% of GFAP⁺, 18% and 14% of Iba1 ⁺, or 3% and 7% of NeuN⁺ cells were edited among astrocytes, microglia, or neurons with Cas9 or Cpf1 (FIGS. 12b and 12d, right panels). Taken together, these results demonstrate that CRISPR-Gold-delivered Cas9 or Cpf1 RNPs can induce deletion of target genes in the major cell types of the brain, including astrocytes, microglia, and neurons.

Materials. Oligonucleotides were purchased from Integrated DNA Technologies (IDT, Coralville, Iowa). Gold nanoparticles (60 nm) were purchased from BBI Solutions (Cardiff, UK). Sodium citrate and 4-(2-hydroxyethyl) piperazine-1-ethanesulfonate (HEPES) were purchased from Mandel Scientific (Guelph, ON). Sodium silicate was purchased from Sigma-Aldrich (St. Louis, Mo.). Phusion High-Fidelity DNA Polymerase was purchased from NEB (Ipswich, Mass.). The Megascript T7 kit, the Megaclear kit, the PageBlue solution, the propidium iodide, and the PureLink Genomic DNA kit were purchased from ThermoFisher (Waltham, Mass.). Mini-PROTEAN TGX Gels (4-20%) were purchased from Bio-Rad (Hercules, Calif.). DMEM media, non-essential amino acids, penicillin-streptomycin, DPBS, and 0.05% trypsin were purchased from Life Technologies (Carlsbad, Calif.). Amicon Ultra-4 30 kDa was purchased from EMD Millipore (Germany). Cas9 proteins with an N-terminal 6×His-tag and two SV40 nuclear localization signal (NLS) peptides at the C-terminus and Cpf1 proteins were purchased from Macrolab UC Berkeley. The Poly (PAsp(DET)) was a gift (Kim, H. J. et al. J Control Release 145, 141-148 (2010); and Miyata, K. et al. J Am Chem Soc 130, 16287-16294 (2008)). The Sytox Red Dead Cell Stain (Excitation/Emission (nm): 640/658) and the SuperScript III First-Strand Synthesis kit were purchased from Invitrogen (Carlsbad, Calif.).

Antibodies. The mouse monoclonal RFP antibody (6G6) was purchased from ChromoTek; the chicken polyclonal GFP antibody (GFP-1020) from Ayes Labs (Tigard, Oreg.); the rabbit polyclonal GFAP antibody (AB5804) and the mouse monoclonal NeuN antibody (MAB377) from Millipore (Burlington, Mass.) the rabbit polyclonal Iba1 antibody (019-19741) from Wako Chemicals (Richmond, Va.); and the rabbit polyclonal mGluR5 antibody (AGC-007) from Alomone Labs (Israel). The goat anti-mouse IgG2a-Cy3, goat anti-chicken-Cy2, goat anti-rabbit-Cy5, goat anti-mouse IgG1-Cy5, and donkey anti-rabbit IgG-Alexa Fluor 647 antibodies were purchased from Jackson ImmunoResearch Laboratories, Inc (West Grove, Pa.).

Synthesis of CRISPR-Gold. Gold nanoparticles (GNPs) (60 nm in diameter, 450 nM) were reacted with a 5′ thiol modified single stranded oligonucleotide (DNA-SH), 26 bases in length (/5ThioMC6-D/GAAATATGCCAGAAATATCTGTCAGA, (SEQ ID NO: 10); 200 μM), which had no sequence specificity. The reaction was performed in an Eppendorf tube in 160 ul of nuclease-free water. The 100 mM sodium citrate solution (pH 3.5, 40 μL) was added to the reaction, and the reaction was allowed to proceed overnight (Zhang, X., Servos, M. R. & Liu, J. J Am Chem Soc 134, 7266-7269 (2012)). Unconjugated DNA-SH was removed by centrifugation at 3,000 g for 10 min, and was washed two times with 20 mM HEPES buffer. The GNP-DNA solution was stored at 4° C. until further use. CRISPR-Gold was synthesized using a layer-by-layer method. Cas9 or Cpf1 (50 pmole in 10 μL) and gRNAs (50 pmole gRNA in 10 μL) were mixed in 80 μL of Cas9/Cpf1 buffer (50 mM HEPES (pH 7.5), 300 mM NaCl, and 10% (vol/vol) glycerol) for 5 min at RT, and this solution was then added to the GNP-DNA solution (0.45 pmole of GNP), generating GNP-Cas9/Cpf1 RNP. Freshly diluted sodium silicate (6 mM, 2 μL) was added to the GNP-Cas9 RNP solution and incubated for 5 min at RT. The mixture was then centrifuged using an EMD Millipore Amicon Ultra-4 30 kDa at 3,000 rpm for 5 min to remove unbound molecules. The recovered GNP-Cas9 RNP-silicate was mixed with 5 μg of PAsp(DET) solution and incubated for 5 min at RT to form the last layer of CRISPR-Gold right before treatment.

Gel electrophoresis to test Cas9 and Cpf1 RNPs loading to CRISPR-Gold. Strong non-covalent interactions allow the interaction of GNP-DNA with Cas9 and Cpf1 RNPs. Gel analysis was conducted to visualize Cas9 and Cpf1 RNPs loading on CRISPR-Gold. CRISPR-Gold was synthesized with the above method with Cas9, sgRNA Ai9 L, and sgRNA_Ai9_R as well as Cpf1 and crRNA_yfp. The synthesized CRISPR-Gold was purified using a Vivaspin 300 kDa concentrator at 3,000 rpm for 5 min to remove unbound molecules, and then one step of washing was additionally conducted. Each sample collected before and after the purification was analyzed with gel electrophoresis using a 4-20% Mini-PROTEAN TGX Gel (Bio-Rad), stained with SYBR Green (ThermoFisher). Additionally, the same gel was stained with Coomassie Blue to visualize the Cas9 and Cpf1 proteins. Images were taken with a ChemiDoc MP using the ImageLab software (Bio-Rad).

Animal care and use. Ai9 (in C57BL/6J background), Thy1-YFP (in C57BL/6J background), mdx, wild-type (in FVB background), and Fmr1 KO (in FVB background) mice were obtained from Jackson Laboratory.

Primary culture of hippocampal neurons from wild-type FVB mice. Hippocampal neurons isolated from embryonic day 17 mouse brains (wild-type FVB mice) were plated at a density of 1-3×10⁵ cells/well as described previously (Fu, W. Y. et al. Nat Neurosci 10, 67-76 (2007)). Cells were kept at 37° C. in a humidified, CO₂-controlled (5%) incubator. Primary cultured hippocampal neurons were cultured for 7 days and were treated with either neurobasal media only or CRISPR-Gold complexes including RNPs (Cas9: 25 pmole and sgRNAs: 25 pmole) with 2.5 μg of PAsp(DET) added in neurobasal media to test electrophysiological properties and toxicity. Fourteen days after CRISPR-Gold treatment, the cells were stained with SYTOX-Red and fixed with 4% PFA, followed by phalloidin-Alexa488 staining with DAPI.

Whole cell recording. 10-14 days after CRISPR-Gold treatment, primary cultured hippocampal neurons were patched and recorded. The extracellular solution contained (in mM) 124 NaCl, 2 KCl, 2 MgSO₄, 1.25 NaH₂PO₄, 2 CaCl₂, 26 NaHCO₃, 10 D-dextrose and 0.4 Vitamin C. The recordings were conducted at room temperature with an internal solution of (in mM) 120 potassium gluconate, 20 KCl, 2 MgCl₂, 10 HEPES, 2 ATP, 0.25 GTP and 0.1 EGTA (pH=7.4). Pyramidal cells were identified at 60× magnification using a water-submersible objective and DIC/infrared optics on a BX51WI Olympus microscope. Recordings were conducted at 25° C. Recordings of voltage used bridge balance compensation via the amplifier (HEKA, EPC10). If slow capacitance changed by more than 20% during recordings, the cell was excluded from further analysis. A hardware filter of 3 kHz was used for data collection. Input resistance was measured using a -20 pA current injection from resting voltage. Action potentials were evoked from a holding current to maintain −80 mV and spikes were generated by rectangular current injection (1 second duration, 200 pA). Action potential frequency was measured 3 min after break-in into the whole-cell.

Primary culture of fibroblasts from Ai9 mice. Primary fibroblasts were obtained from the liver, muscle or tail of Ai9 mice. Collagenase-treated tissues were minced with scaffold and digested in a collagenase and trypsin mixture (Khan, M. & Gasser, S. J Vis Exp (2016)). The cells were plated in 10 cm culture dishes with the culture medium. Cells that were not firmly attached were removed during media changes that were conducted every 24 hr. Fibroblasts were passaged with Accutase, and transfection with Cas9 or Cpf1 was conducted with fibroblasts within 14 days of culture.

Nucleofection. Cells were detached by accutase, spun down at 600 g for 3 min, and washed with PBS. Nucleofection was conducted using an Amaxa 96-well Shuttle system following the manufacturer's protocol, using 10 μL of Cas9 RNPs (Cas9: 100 pmole, gRNAs: 120 pmole) or Cpf1 RNPs (Cpf1: 100 pmole, crRNAs: 120 pmole) (Lin, S., Staahl, B. T., Alla, R. K. & Doudna, J. A. Elife 3, e04766 (2014)). 10⁵ cells were transfected using the EH-100 Lonza program. After nucleofection, 500 μL of media was added and the cells were incubated at 37° C. in tissue culture plates. The cell culture media was changed the next day, and the cells were then incubated for 7 days before flow cytometry analysis.

Flow cytometry analysis & fluorescence microscopy. Flow cytometry was used to quantify the expression levels of YFP in YFP-HEK cells or expression levels of tdTomato in primary Ai9 fibroblasts after transfecting with Cas9 or Cpf1. The cells were analyzed 7 days after transfections. The cells were washed with PBS and detached by accutase. YFP and tdTomato expression was quantified using BD LSR Fortessa X-20 and Guava easyCyte™ and analysis was conducted with FlowJo.

Immunostaining. Two weeks after the stereotaxic injection of 1-2 month old adult mice, the mice were anesthetized by isoflurane and perfused through the left ventricle with ice-cold PBS followed by 4% paraformaldehyde in PBS. The brains were post-fixed for 4 hr in 4% PFA, washed once with PBS, and then moved to 30% sucrose in PBS at 4° C. Hippocampal sections from the mice were obtained by cryostat (CM3050S; Leica Microsystems, Bannockburn, Ill.). Prior to sectioning, brains were cryoprotected by incubation in 30% sucrose, embedded in the O.C.T. compound, and frozen/stored at −80° C. until slicing. Slices were cut on the coronal plane at 20 μm, mounted on glass slides, and stored at 4° C. The sections to be immunostained were washed three times in PBS. Antigen retrieval was performed by steaming in a citrate buffer (0.294% sodium citrate, 0.05% Tween 20 in ddH₂O, pH 6.0) for 15 min with subsequent cooling over ice for 10 min. The sections were rinsed in PBS and blocked (5% goat serum, 0.2% Triton X-100 in PBS) overnight at 4° C. The sections were incubated in the same blocking solution with primary antibodies at room temperature for 1 hr. The sections were washed in PBS prior to incubation with secondary antibodies for 2 hr. After rinsing once more in PBS, the sections were mounted in Prolong Gold Antifade Reagent with DAPI and imaged using a Zeiss confocal microscope.

Analysis and statistics for immunostaining. Quantification of fluorescence was performed on images taken with the same exposure times and within the same experiment. To determine relative YFP intensities in Thy1-YFP mice, a defined region of interest (ROI), which was the same size for all images analyzed, was traced in the hippocampus using the Image J software (NIH). To quantify the YFP gene knockdown, YFP⁺ cells were also counted and normalized to DAPI⁺ cells in a defined ROI. To determine the % of edited cells or to present the number of DAPI⁺ cells in Ai9 mice, tdTomato⁺ cells were counted and normalized by the number of DAPI⁺ cells or the number of DAPI⁺ cells were presented itself (analyzed from a defined ROI, which was the same size for all images analyzed to compare). To determine the % of cell types among edited cells, GFAP⁺, Iba1⁺, or NeuN⁺ cells were counted in only tdTomato⁺ cells. The % of the tdTomato⁺ cells among the cell types was also analyzed by counting GFAP⁺, Iba1⁺, or NeuN⁺ cells co-stained with tdTomato among the total GFAP⁺, Iba1⁺, or NeuN⁺ cells. Each cell marker was stained with tdTomato and analyzed independently. To compare the uninjected group and the injected group, the student's unpaired t-test (two-tailed) was used. For mGluR5 immunostaining analysis, the total mGluR5⁺ cells were counted and normalized by DAPI⁺ cells. The student's unpaired t-test was used for the statistical analysis. All the statistics showed that variances are similar between the groups that are being statistically compared. No statistical methods were used to pre-determine sample sizes, but sample sizes are similar to those generally employed in the field. Sample size (n) is indicated in each figure legend. The injection side was randomized (left or right side of the brain) for each experiment.

### Example 2: Nanoparticle Delivery of CRISPR into the Brain Rescues Increased Repetitive Behaviors in the Mouse Model of Fragile X Syndrome

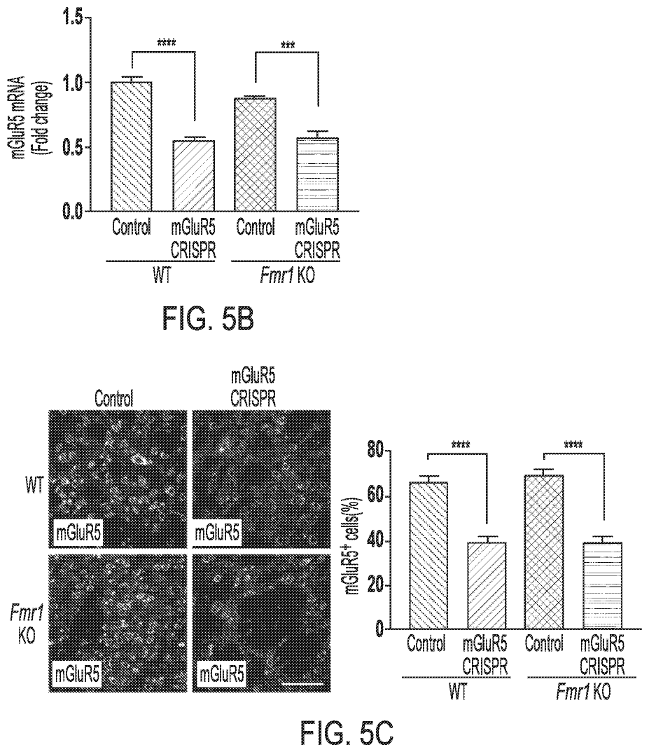

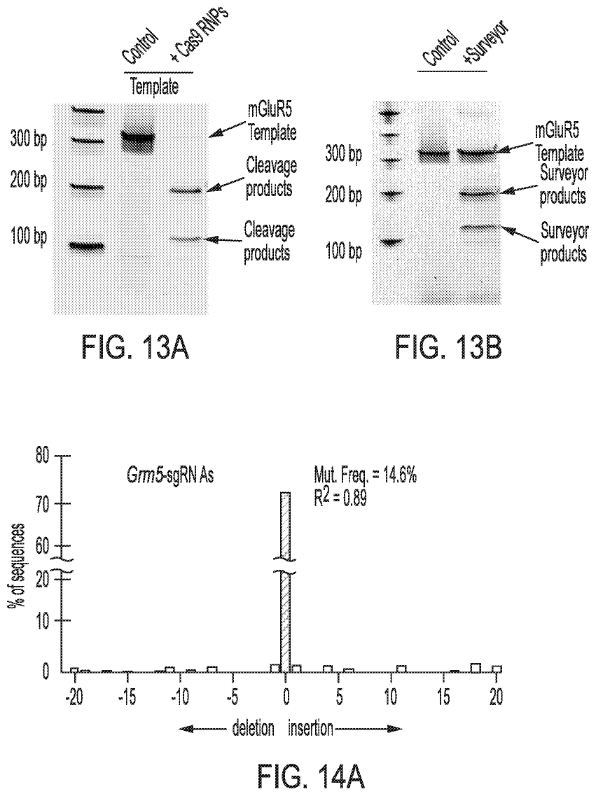

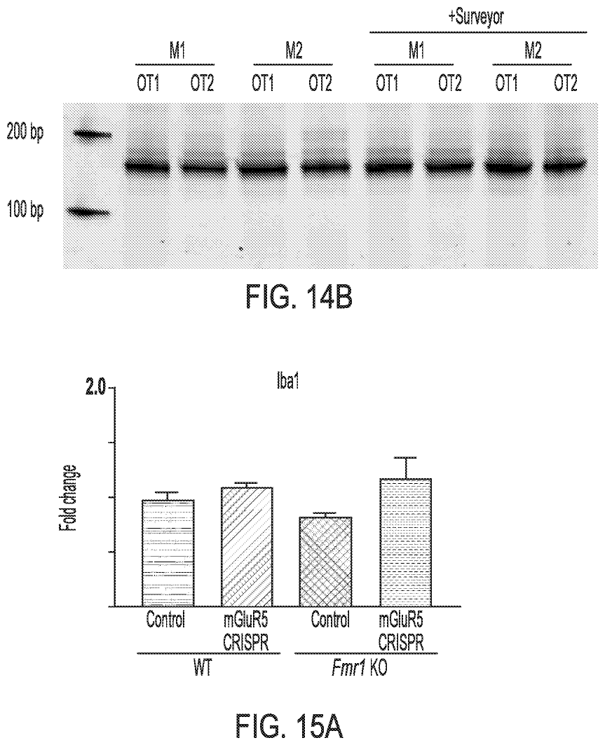

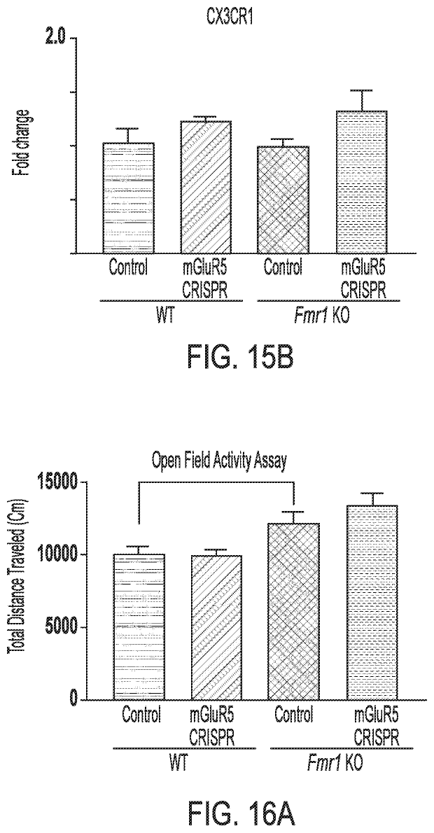

These results described herein indicate that CRISPR-Gold has the potential to treat numerous brain disorders given its ability to edit genes in the brains of adult animals. The mGluR5 gene (Grm5) was selected as a target for CRISPR-Gold-based therapeutic gene editing because a wide number of studies have demonstrated that exaggerated mGluR5 signaling can generate FXS pathophysiology. Therefore, knocking out the mGluR5 gene through non-viral CRISPR gene editing in specific regions of the brain may be a therapeutic way to treat FXS in patients. However, it is unclear if exaggerated mGluR5 signaling-mediated FXS phenotypes are caused by focal overactivation of mGluR5 signaling (versus global), and if so, which parts of the brain need to have the mGluR5 gene deleted to rescue from the specific behavioral phenotypes. Furthermore, the delivery challenges associated with gene editing in the brain with Cas9 RNPs need to be solved to be used as treatments of brain disorders. To address these two unresolved issues in therapeutic brain gene editing, CRISPR-Gold Cas9-sgRNA RNPs targeting the mGluR5 gene (Grm5) were generated, and investigated for their ability to knock out the mGluR5 gene in vivo and rescue mice from the behavioral phenotypes of FXS using Fmr1 KO mice, a mouse model of FXS. It was confirmed that CRISPR-Gold-mediated mGluR5 gene editing is successful, in vitro and in cells, as shown in FIG. 13. Next, stereotaxically injected either saline vehicle (Control) or CRISPR-Gold targeting the mGluR5 gene (mGluR5-CRISPR) into the striatum of WT or Fmr1 KO mice was carried out to see if CRISPR-Gold could knock out the mGluR5 gene in vivo in the striatum, after a direct local injection (FIG. 5a). By tracking of indels by decomposition (TIDE) analysis, 14.6% in frequency of mGluR5 gene mutations were detected (FIG. 14a), and there were no significant off targets observed as shown in FIG. 14b. As a result, the mRNA levels or the protein levels of mGluR5 were reduced by about 40-50% both in WT and Fmr1 KO mice, which was confirmed by reverse transcription (RT)-qPCR (FIG. 5b) and immunostaining analysis (FIG. 5c). There were no significant symptoms of increased immune response measured by mRNA levels of microglia markers in mGluR5-CRISPR-treated brains (FIG. 15).

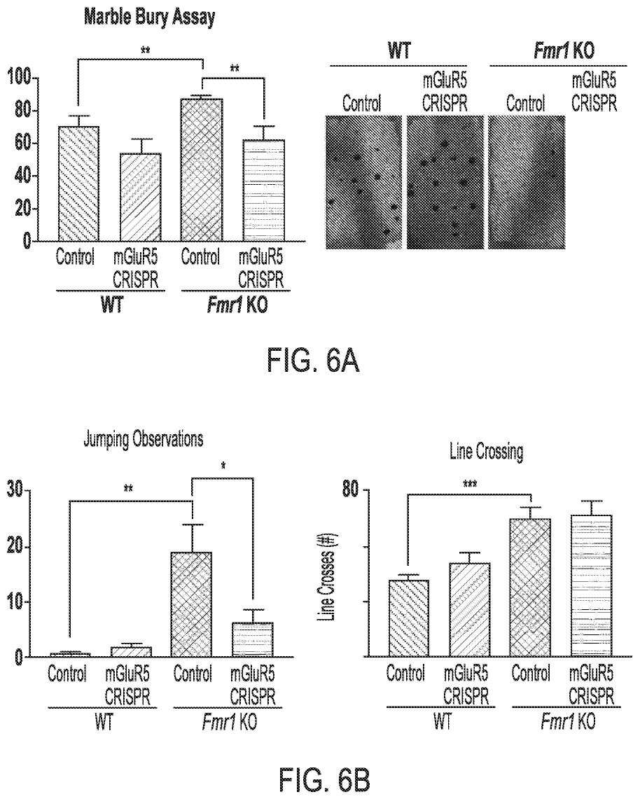

Commonly known repetitive behaviors of mice with autistic phenotypes such as Fmr1 KO mice include excessive digging behavior, which can be observed in the marble bury assay, and increased jumping, which can be observed during empty cage observations (Sukoff Rizzo, S. J. & Crawley, J. N. Annu Rev Anim Biosci 5, 371-389 (2017)). Given that the striatum is an important brain region in mediating repetitive behaviors, experiments were performed to test if knocking out the mGluR5 gene by CRISPR-Gold delivery of Cas9 RNPs can rescue the exaggerated repetitive behaviors shown in Fmr1 KO mice. FIG. 6 demonstrates the effect of the saline control or mGluR5-CRISPR injections into the striatum on repetitive behaviors in WT and Fmr1 KO mice. In the marble bury assay, Fmr1 KO mice injected with saline buried significantly more marbles than WT mice, but injection with mGluR5-CRISPR into the striatum significantly rescued the excessive digging phenotype of Fmr1 KO mice back to normal, while having no significant effect on WT mice (FIG. 6a,). Videos of marble bury assay were taken and complied. The data showed a WT mouse injected with saline (WT Control: top left video), an Fmr1 KO mouse injected with saline (Fmr1 KO Control: bottom left video), a WT mouse injected with mGluR5-CRISPR (WT mGluR5-CRISPR: top right video), and an Fmr1 KO mouse injected with mGluR5-CRISPR (Fmr1 KO mGluR5-CRISPR: bottom right video). The four videos, each 30 minutes in length, were recorded with DVC Full HD Camcorders at a 640×480 resolution. Twenty marbles were placed equidistant from each other on 3 cm of bedding inside a standard home cage. The mouse was placed inside the cage with a clear plastic covering and allowed to freely roam for 30 minutes before removal. All marbles with surfaces more than one-third visible were circled at the end of the video, indicating they were unburied. Marbles buried by WT mice are colored green while those buried by Fmr1 KO mice are colored red. The individual videos are played at 12× their normal speed. The software used to edit the videos included Movie Studio Platinum 13.0, Movavi Video Converter, and Microsoft PowerPoint 2016.

Likewise, the same effect was shown in jumping behavior in Fmr1 KO mice, where those injected with mGluR5-CRISPR showed a phenotype much more comparable to WT mice as compared to Fmr1 KO mice injected with saline (FIG. 6b). Videos were taken of empty cage observations and complied. The data showed a WT mouse injected with saline (WT Control: top left video), an Fmr1 KO mouse injected with saline (Fmr1 KO Control: bottom left video), a WT mouse injected with mGluR5-CRISPR (WT mGluR5-CRISPR: top right video), and an Fmr1 KO mouse injected with mGluR5-CRISPR (Fmr1 KO mGluR5-CRISPR: bottom right video). The four videos, each originally 20 minutes in length, were recorded with DVC Full HD Camcorders at a 640×480 resolution. The first 10 minutes were reserved for habituation and excluded from analysis. The remaining 10 minutes worth of observations were selected for scoring. The left counter in each video, “Jumps,” increases by one each time the mouse jumped onto the nozzle or simultaneously lifted both of its back feet off the cage floor. The right counter in each video, “Crosses,” increases by one each time 75% of the mouse's body, excluding the tail, crossed the center of the cage. The center is marked by the colored line (green indicating WT and red indicating Fmr1 KO mice) down the middle of each video. The individual videos are played at 4× their normal speed. The software used to edit the videos included Movie Studio Platinum 13.0, Movavi Video Converter, and Microsoft PowerPoint 2016.

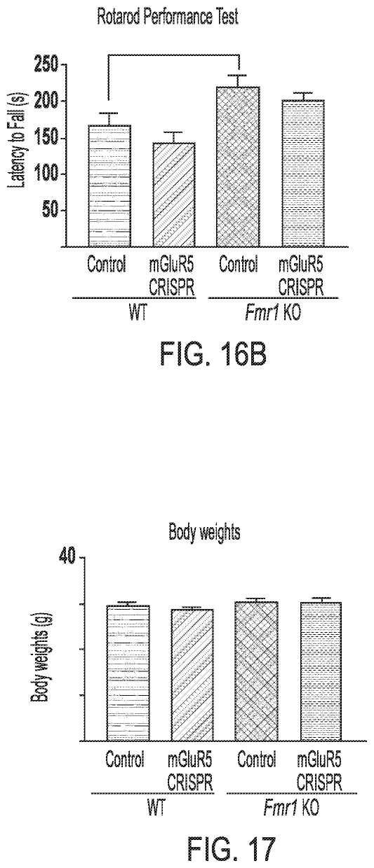

To determine if the rescue of excessive digging or jumping behaviors were the consequences of potential reduced hyperlocomotor activities in Fmr1 KO mice (Ding, Q., Sethna, F. & Wang, H. Behav Brain Res 271, 72-78 (2014)) by mGluR5-CRISPR, locomotor activities in mice were also assessed. This can be observed by line crossing, which can be observed during empty cage observations (Sungur, A., Vörckel, K. J., Schwarting, R. K. & Wöhr, M. J Neurosci Methods 234, 92-100 (2014)), the distance that a mouse travels during an open field activity assay (Ding, Q., Sethna, F. & Wang, H. Behav Brain Res 271, 72-78 (2014)), and how long it takes for a mouse to fall in the accelerated rotarod performance test (Graham, D. R. & Sidhu, A. J Neurosci Res 88, 1777-1783 (2010)). Five weeks after injection, mice were weighed to check for any potential side-effects of treatment. While there was a significant difference between WT and Fmr1 KO mice when it comes to these phenotypes, there was no significant change of locomotor activity by mGluR5 reduction of Fmr1 KO mice in any of these three experiments performed (FIG. 6B, right panel and FIG. 16), nor were significant body weight changes found (FIG. 17), suggesting that the exaggerated mGluR5 signaling in the striatum does not mediate the hyperlocomotor activity shown in Fmr1 KO mice. In other words, increased repetitive behaviors were specifically rescued by mGluR5-CRISPR treatment to the striatum of Fmr1 KO mice. Taken together, these results demonstrate that rescuing of specific behavioral phenotypes in an autism mouse model is possible with gene editing using non-viral delivery of CRISPR into a local brain region.

In vitro T7 transcription of sgRNA and crRNA. The DNA templates for in vitro transcription of sgRNAs and crRNAs (sgRNAs for Cas9 and crRNAs for Cpf1) were prepared by PCR. The sequence of the template and primers are listed in Table 1 and Table 2. Two sequences (left and right) of gRNAs were used for Ai9 targeting experiments. A single sequence of sgRNAs or crRNAs for YFP gene (yfp) or sgRNAs for mGluR5 gene (Grm5) targeting was used. PCR amplification was performed with Phusion Polymerase according to the manufacturer's protocol. RNA in vitro transcription was performed with the MEGAscript T7 kit (ThermoFisher) and purification of the resulting RNA was conducted using the MEGAclear kit, following the manufacturer's protocol. The transcribed sgRNAs and crRNAs were eluted into 20 mM HEPES buffer. The concentration of RNAs was determined with a Nanodrop 2000 and the final gRNA products were stored at −80° C. for subsequent experiments. Ai9 sgRNA target sequences were chosen based on Tabebordbar et al.³ and other target sequences were designed in house.

TABLE 1
List of target sequences for gRNAs (5′ to 3′)

| gRNA | Target sequences | SEQ ID NO: |
|---|---|---|
| crRNA_Ai9_L | AATATAACTTCGTATAATGTATGC | 1 |
| crRNA_Ai9_R | TCCAAACTCATCAATGTATCTTAT | 2 |
| sgRNA_Ai9_L | AAAGAATTGATTTGATACCG | 3 |
| sgRNA_Ai9_R | GTATGCTATACGAAGTTATT | 4 |
| crRNA_yfp | CGTCGCCGTCCAGCTCGACCAGGA | 5 |
| sgRNA_yfp | GTAGCCGAAGGTGGTCACGA | 6 |
| sgRNA_Grm5 | GGACTGACAGAATCAACAGA | 7 |

TABLE 2
crRNA and sgRNA backbone sequences (5′ to 3′)

| | | SEQ ID NO: |
|---|---|---|
| crRNA | TAATACGACTCACTATATAATTTCT | 8 |
| backbone | ACTCTTGTAGATNNNNNNNNNNNN | |
| | NNNNNNNNNNNN | |
| sgRNA | TAATACGACTCACTATAGNNNNNN | 9 |
| backbone | NNNNNNNNNNNNNNGTTTTAGAG | |
| | CTAGAAATAGCAAGTTAAAATAAG | |
| | GCTAGTCCGTTATCAACTTGAAAA | |
| | AGTGGCACCGAGTCGGTGCTTTTT | |
| | T | |

TABLE 3
List of crRNA sequences (5′ to 3′)

| | | SEQ ID |
|---|---|---|
| Name | Sequences | NO: |
| crRNA_Ai9_L | AAUAUAACUUCGUAUAAUGUAUGC | 33 |
| crRNA_Ai9_R | UCCAAACUCAUCAAUGUAUCUUAU | 34 |
| sgRNA_YFP | CGUCGCCGUCCAGCUCGACCAGGA | 35 |

The conserved 5′ sequence of crRNA can be UAAUUUCUACUCUUGUAGAU (SEQ ID NO: 36).

TABLE 4
List of sgRNA sequences (5′ to 3′)

| | | SEQ ID |
|---|---|---|
| Name | Guide sequences | NO: |
| sgRNA_Ai9_L | AAAGAAUUGAUUUGAUACCG | 33 |
| sgRNA_Ai9_L | GUAUGCUAUACGAAGUUAUU | 34 |
| sgRNA_YFP | GUAGCCGAAGGUGGUCACGA | 35 |
| sgRNA_grm5 | GGACUGACAGAAUCAACAGA | 36 |

The conserved 3′ sequence of sgRNA can be GUUUUAGAGCUAGAAAUAGCAAGUUAAAAUAAGGCUAGUCCGUUAUCAACUU GAAAAAGUGGCACCGAGUCGGUGCUUUU (SEQ ID NO: 37).

Culture of YFP-expressing HEK cells. YFP-HEK cells were generated by infection of HEK293T cells (from UC Berkeley) with a YFP-containing lentivirus, and clonal selection was done for cells expressing YFP. YFP-HEK cells were cultured in the culture medium (DMEM with 10% FBS, lx MEM, non-essential amino acids, and 100 μg/mL Pen-Strep). The cells have been tested for mycoplasma contamination and the result was negative. Nucleofection was conducted on the YFP-HEK cells.

In vitro and in cell cleavage assays. The mGluR5 template was PCR-amplified (mGluR5 Forward: CCTTAATGCACCACTCAGCA (SEQ ID NO: 11), mGluR5 Reverse: GGCTTCCACTCTCTGAATGC (SEQ ID NO: 12)) from mouse genomic DNA. For in vitro cleavage assays, the template DNA was incubated with Grm5 sgRNA and Cas9 proteins (Grm5 Cas9 RNPs) in a 1.5 ml tube. Gel running was performed to see cleavage of the template. For cleavage assays in the myoblasts from mdx mice, Grm5 Cas9 RNPs was introduced into the cell with electroporation. The mGluR5 gene was then PCR-amplified from the myoblast and a surveyor assay was conducted to check target gene editing.

Stereotaxic injection of CRISPR-Gold into the mouse brain. 1-2 month old adult mice were anesthetized by intraperitoneal (i.p.) injection of 100 mg/kg ketamine and 10 mg/kg xylazine. Preemptive analgesia was given (Buprenex, 1 mg/kg, i.p.). Craniotomy was performed according to approved procedures, and 2 μl of CRISPR-Gold (Cas9: 50 pmole and sgRNAs: 50 pmole, or Cpf1: 50 pmole and crRNAs: 50 pmole) with 5 μg of PAsp(DET) for Thy1-YFP or Ai9 mice was injected into a single hemisphere of the striatum and/or the dorsal dentate gyrus of the hippocampus. The uninjected contralateral side was used as a control. For stereotaxic injection with mGluR5-CRISPR, 2 p1 of saline (Control) or CRISPR-Gold loaded with Cas9-mGluR5 RNPs (Cas9: 50 pmole and sgRNAs: 50 pmole) was injected into the striatum of both hemispheres of WT or Fmr1 KO mice. Injection was given separately into three spots in each hemisphere with a 0.4 mm interval. The incision was clipped and proper post-operative analgesics were administered for 6 days following surgery.

Deep sequencing analysis of CRISPR-Gold-treated brain tissue. The target sequences of the genomic region were amplified by PCR using Phusion High-Fidelity Polymerase according to the manufacturer's protocol. Target genes were amplified first with primer sets and then amplified again with deep sequencing primers listed in Table 5. The amplicons were purified using the ChargeSwitch PCR clean-up kit (ThermoFisher). Lastly, PCR with barcode primers was conducted to attach Illumina adapters for deep sequencing. PCR clean-up was performed one additional time. The Berkeley Sequencing facility performed DNA quantification using a Qubit 2.0 Fluorometer (Life Technologies, Carlsbad, Calif.). BioAnalyzer for size analysis and qPCR quantification followed. The library was sequenced with the Illumina HiSeq2500 in the Vincent Coates Genomic Sequencing Laboratory at UC Berkeley. The analysis was conducted using the CRISPR Genome Analyzer (Güell, M., Yang, L. & Church, G. M. Bioinformatics 30, 2968-2970 (2014)).

TABLE 5
Deep sequencing primers

| | | SEQ |
|---|---|---|
| | | ID |
| Name | Primer Sequence | NO: |
| YFP_F | TGAGCAAGGGCGAGGAGCTGT | 13 |
| YFP_R | GTCCTCCTTGAAGTCGATGCCCTT | 14 |
| YFP_Cas9_ | TCTTGTGGAAAGGACGAAACACCGTCATC | 15 |
| Deep_F | TGCACCACCGGCAAGCT | |
| YFP_Cas9_ | TCTACTATTCTTTCCCCTGCACTGTCCGTCG | 16 |
| Deep_R | TCCTTGAAGAAGATGGT | |
| YFP_Cpf1_ | TCTTGTGGAAAGGACGAAACACCGTGAGC | 17 |
| Deep_F | AAGGGCGAGGAGCTGTTCA | |
| YFP_Cpf1_ | TCTACTATTCTTTCCCCTGCACTGTTTGCCG | 18 |
| Deep_R | TAGGTGGCATCG | |

TIDE assay. mGluR5 target gene was amplified by PCR using Phusion Polymerase. The PCR amplicon was sent to Quintara Bioscience for sequencing. The sequencing result was analyzed with TIDE software to quantify indel mutation efficiency (https://tide-calculator.nki.nl/) (Brinkman, E. K., Chen, T., Amendola, M. & van Steensel, B. Nucleic Acids Res 42, e168 (2014)).

Off-target analysis. Off-target prediction was conducted using Cas9-OFFinder (http://www.rgenome.net/cas-offinder/). The top two off-target sites had more than ten base matches. Genomic DNA extracted from mouse brains that were injected with mGluR5-CRISPR (CRISPR-Gold complex) was used to amplify two off-target sites. A surveyor assay and polyacrylamide gel electrophoresis were conducted to detect cleaved products.

RNA extraction from mouse brains and reverse transcription (RT)-qPCR. Mice were perfused with ice-cold PBS at 11 weeks after injection. Brains were cut into 1 mm sections by using a brain slicer matrix (Zivic instrument) around the injection sites. The brain slices were washed with ice-cold PBS and the injection region (1 mm thick×mm wide×mm long) was cut out. After adding 800 μl of TRIzol, the brain slices were homogenized, treated with 160 μl of chloroform, and centrifuged for 15 min at 4° C. The aqueous phase of the sample was removed by pipet and 400 μl of 100% isopropanol was added. After being centrifuged for 10 min, the supernatant was removed from the tube, and then the pellet was washed with 75% ethanol and centrifuged for 5 min. Afterwards, the supernatant was removed and the pellet was dissolved in DNase- and RNase-free water. One μg of RNA was reverse-transcribed using a SuperScript III First-Strand Synthesis kit (Invitrogen). RT-qPCR analysis was performed using PowerUp SYBR Green Master Mix (Applied Biosystems) with the primers listed in Table 6. The relative expression from RNA samples was analyzed using the 2^{-ΔΔCT} method. Values were normalized by the PPIA housekeeping gene expression.

TABLE 6
RT-qPCR primers (mouse)

| | | SEQ |
|---|---|---|
| | | ID |
| Gene | Primer | NO: |
| Grm5 (mGluR5) F | GCTGTGAGATAAGAGATTCCTGC | 19 |
| Grm5 (mGluR5) R | ACTCCCACTATGGGTTTCTTGG | 20 |
| Iba1 (Iba1) F | ATCAACAAGCAATTCCTCGATGA | 21 |
| Iba1 (Iba1) R | CAGCATTCGCTTCAAGGACATA | 22 |
| Cx3cr1 (CX3CR1) F | GAGTATGACGATTCTGCTGAGG | 23 |
| Cx3cr1 (CX3CR1) R | CAGACCGAACGTGAAGACGAG | 24 |
| Ppia (PPIA) F | GAGCTGTTTGCAGACAAAGTTC | 25 |
| Ppia (PPIA) R | CCCTGGCACATGAATCCTGG | 26 |

Behavioral test sequence and statistics. Behavior tests were performed on mice at 2 weeks post injection in the following order: empty cage observations, marble burying assay, open field activity assay, and rotarod performance test, followed by weighing and perfusion of all animals (Table 7). A total of 47 mice (n=11-12 per group) were used as a cohort. Identification of each animal was determined after testing to ensure that the experimenter remained blind to the genotype or treatment of the test subject. Unless otherwise noted, data were analyzed by one-way ANOVA using GraphPad's Prism7 software.

TABLE 7
Behavioral test sequence

| Schedule | Test | Index |
|---|---|---|
| Day 1-2 | Empty cage observations | Jumping (#), |
| | | Line crossing (#) |
| Day 4-5 | Marble burying assay | Marbles buried (%) |
| Day 7-8 | Open field activity assay | Total distance (cm) |
| Day 10-11 | Rotarod performance test | Latency to fall (sec) |

Empty cage observations. The test was performed as previously described (McFarlane, H. G. et al. Autism-like behavioral phenotypes in BTBR T+tf/J mice. Genes Brain Behav 7, 152-163 (2008)) with minor modifications. The mice were brought into a testing room with normal lighting 1.5 hr before testing, after which they were then individually placed in a test cage identical to their home cage (30 cm W×19 cm L×13 cm H) without bedding to prevent digging for 10 min of habituation. The cage was recorded with a DV Cam camera for 12 min while the mouse was allowed to freely explore. The last 10 min of the video was analyzed for: line crossing (crossing an imaginary line at the center of the cage) and jumping by investigators who were blind to the genotype or treatment of the test subject.

Marble burying assay. The test was performed as previously described (Spencer, C. M. et al. Autism Res 4, 40-56 (2011)) with minor modifications to evaluate repetitive digging behavior (Thomas, A. et al. Psychopharmacology (Berl) 204, 361-373 (2009)). The mice were brought into the testing room with normal lighting 1.5 hr before testing after which they were individually placed in a cage identical to the test cage (30 cm W×19 cm L×13 cm H) for 30 min of habituation. The test cage was filled with 3 cm of Teklad Sani-Chip bedding, and 20 dark blue marbles (15 mm diameter) were placed in a 5×4 pattern equidistant to each other and the side of the cage. The mice were then placed into the test cage and allowed to freely explore for 30 min. A marble was considered buried if ⅔ of the marble's surface was covered by bedding. The percentage of marbles buried was then scored by 4 different investigators who were blind to the genotype or treatment of the test subject.

Open field activity assay. The open field activity assay was performed as previously described (Bailey, K. R., Pavlova, M. N., Rohde, A. D., Hohmann, J. G. & Crawley, J. N. Pharmacol Biochem Behav 86, 8-20 (2007)) with minor modifications. The mice were brought into the dark testing room with dim red lighting 1 hr prior to the test after which they were then individually placed in a cage identical to their home cage for 30 min. The mice were then placed in the right corner of a clear acrylic chamber and allowed to freely explore for 30 min. Specific measures such as total distance traveled for 30 min were shown for measuring motor activity.

Rotarod performance test. The rotarod apparatus was used to measure locomotor activity (Graham, D. R. & Sidhu, A. J Neurosci Res 88, 1777-1783 (2010)). During the training period, mice were allowed to explore the cylinder of the rotarod for 2 min with constant rotation at a speed of 4 rpm. After 5 min rest, they were put on the rotarod and accelerated to a speed of 4-40 rpm over a period of 300 sec. The latency to fall off of the rotarod within this time period was recorded (up to 300 sec). Mice received 4 trials, and the mean latency to fall off of the rotarod for all 4 trials was combined and calculated.

What is claimed is:

1. A CRISPR-Gold system comprising:
a) a plurality of DNA oligonucleotides conjugated to a gold nanoparticle forming a DNA oligonucleotide-gold nanoparticle (GNP);
b) one or more RNA-guided endonuclease proteins conjugated to one or more guide RNA molecules (gRNA) forming one or more ribonucleoprotein (RNPs); and
c) a biodegradable polymer.

2. The CRISPR-Gold system of claim 1, wherein the one or more RNPs are conjugated to the GNP forming a RNP-GNP complex, and wherein the biodegradable polymer encapsulates the RNP-GNP complex thereby forming the CRISPR-Gold system.

3. The CRISPR-Gold system of claim 1, wherein the gRNA hybridizes with a target sequence of a DNA locus in a cell.

4. The CRISPR-Gold system of claim 1, wherein the gRNA targets and hybridizes with a target sequence and directs the one or more RNA-guided endonuclease proteins to the DNA locus.

5. The CRISPR-Gold system of claim 1, wherein the gRNA sequence is selected from Table 4.

6. The CRISPR-Cas system of claim 1, wherein the one or more RNA-guided endonuclease proteins is a Cas9 protein or a Cpf1 protein.

7. The CRISPR-Cas system of claim 1, wherein the biodegradable polymer is PAsp(DET).

8. The CRISPR-Cas system of claim 3, wherein the cell is a eukaryotic cell.

9. The CRISPR-Cas system of claim 8, wherein the cell is a mammalian or human cell.

10. The CRISPR-Cas system of claim 3, wherein the target sequence of a DNA locus in a cell is fragile X mental retardation 1 (FMRI) gene or metabotropic glutamate receptor 5 (Grm5) gene.

11. A method of modulating expression of a gene in a cell, the method comprising:
a) introducing into the cell a CRISPR-Gold system, comprising:
i) a plurality of DNA oligonucleotides conjugated to a gold nanoparticle forming a DNA oligonucleotide-gold nanoparticle (GNP);
ii) one or more RNA-guided endonuclease proteins conjugated to one or more guide RNA molecules (gRNA) forming one or more ribonucleoprotein (RNPs), wherein the gRNA is complementary to a target nucleic acid sequence comprising the gene; and
iii) a biodegradable polymer.

12. The method of claim 11, wherein the one or more RNPs are conjugated to the GNP forming a RNP-GNP complex, and wherein the biodegradable polymer encapsulates the RNP-GNP complex thereby forming the CRISPR-Gold system.

13. The method of claim 11, wherein the cell produces the gRNA and the gRNA hybridizes with a target sequence of a DNA locus in a cell; wherein the gRNA targets and hybridizes with a target sequence and directs the one or more RNA-guided endonuclease proteins to the DNA locus; and wherein the DNA locus modulates expression of the gene.

14. The method of claim 11, wherein the gRNA sequence is selected from Table 4.

15. A method for introducing into a cell a CRISPR-Gold system comprising:
a) a plurality of DNA oligonucleotides conjugated to a gold nanoparticle forming a DNA oligonucleotide-gold nanoparticle (GNP);
b) one or more RNA-guided endonuclease proteins conjugated to one or more guide RNA molecules (gRNA) forming one or more ribonucleoprotein (RNPs); wherein the gRNA hybridizes with a target sequence of a DNA molecule in a cell; and
c) a biodegradable polymer.

16. The method of claim 15, wherein the one or more RNPs are conjugated to the GNP forming a RNP-GNP complex, and wherein the biodegradable polymer encapsulates the RNP-GNP complex thereby forming the CRISPR-Gold system.

17. The method of claim 15, wherein the gRNA targets and hybridizes with a target sequence and directs the one or more RNA-guided endonuclease proteins to the DNA locus.

18. The method of claim 15, wherein the gRNA sequence is selected from Table 4.

19. A pharmaceutical composition comprising the CRISPR-Gold system of claim 1.

20. The pharmaceutical composition of claim 19, wherein the composition is formulated for systemic or intracranial administration.

21. A method of treating a subject having fragile X syndrome, the method comprising administering to the subject a therapeutically effective amount of the composition of claim 1 or 19, wherein the gRNA is SEQ ID NO: 36.

22. The method of claim 21, further comprising identifying a subject having fragile X syndrome.

23. The method of claim 21, wherein in the fragile X syndrome is caused by increased metabotropic glutamate receptor 5 (mGluR5) signaling.

24. The method of claim 21, wherein the composition is administered into the brain or striatum.

25. A method of treating a subject having fragile X syndrome, the method comprising:
(a) determining mGluR 5 signaling or a mGluR5-mediated behavioral phenotype in the subject; and
(b) administering to the subject a pharmaceutical composition comprising a CRISPR-Gold system comprising one or more RNA-guided endonuclease proteins conjugated to one or more guide RNA molecules (gRNA), wherein the gRNA is SEQ ID NO: 36.

26. A method for targeted genomic modification of Grm5 in mammalian cells, the method comprising administering a CRISPR-Gold nanoparticle, wherein the CRISPR-Gold nanoparticle comprises a guide RNA sequence that targets and hybridizes with a sequence that encodes Grm5.

27. A CRISPR-Gold system for targeted genomic modification of Grm5 in mammalian cells, wherein the system comprises a guide RNA sequence that targets and hybridizes with a sequence that encodes Grm5.

28. The CRISPR-Gold system of claim 26 or 27, wherein the mammalian cells are human cells.

29. A guide RNA (gRNA) molecule that targets one or more nucleotides in a Grm5 molecule.

30. The gRNA molecule of claim 29, wherein the RNA molecule targets a nucleic acid sequence that encodes the Grm5 molecule, wherein the nucleic acid sequence that encodes the Grm5 molecule comprises one or more of: a sequence encoding an amino acid sequence of the Grm5 molecule, a sequence encoding the amino acid sequence of the Grm5 molecule comprising non-translated sequence, or a sequence encoding the amino acid sequence of the Grm5 molecule comprising non-transcribed sequence.

31. The gRNA molecule of claim 30, wherein the nucleic acid that encodes the Grm5 molecule corresponds to SEQ ID NO: X.

32. The gRNA molecule of any of claims 29-31, wherein the gRNA molecule is configured to provide a Cas9 molecule-mediated cleavage event in the nucleic acid that encodes the Grm5 molecule.

33. The gRNA molecule of any of claims 29-32, wherein the gRNA molecule:
targets the sequence encoding an amino acid sequence of the Grm5 molecule;
is configured to provide a Cas9 molecule-mediated cleavage event in the sequence encoding an amino acid sequence of the Grm5 molecule; or
comprises a targeting domain configured to provide a Cas9 molecule-mediated cleavage event in the sequence encoding an amino acid sequence of the Grm5 molecule.

34. A method of treating a subject, the method comprising contacting a cell or a subject with an effective amount of a gRNA molecule of any of claims 29-32.

35. The method of claim 34, further comprising altering the sequence of the target nucleic acid.

36. The method of claim 34 or 35, wherein the cell is a vertebrate, mammalian or human cell.

37. The method of claim 36, wherein the cell is a brain cell.

38. A pharmaceutical preparation comprising a gRNA molecule of any of claims 29-25.

39. Compositions and methods described herein.


SEQUENCE LISTING (37 sequences)

SEQ ID NO: 1 - DNA, 24 bp, Artificial Sequence
  Synthetic contruct
aatataacttcgtataatgtatgc

SEQ ID NO: 2 - DNA, 24 bp, Artificial Sequence
  Synthetic contruct
tccaaactcatcaatgtatcttat

SEQ ID NO: 3 - DNA, 20 bp, Artificial Sequence
  Synthetic construct
aaagaattgatttgataccg

SEQ ID NO: 4 - DNA, 20 bp, Artificial Sequence
  Synthetic construct
gtatgctatacgaagttatt

SEQ ID NO: 5 - DNA, 24 bp, Artificial Sequence
  Synthetic construct
cgtcgccgtccagctcgaccagga

SEQ ID NO: 6 - DNA, 20 bp, Artificial Sequence
  Synthetic construct
gtagccgaaggtggtcacga

SEQ ID NO: 7 - DNA, 20 bp, Artificial Sequence
  Synthetic construct
ggactgacagaatcaacaga

SEQ ID NO: 8 - DNA, 61 bp, Artificial Sequence
  Synthetic construct
  misc_feature (38)..(61): n is a, c, g, or t
taatacgactcactatataatttctactcttgtagatnnnnnnnnnnnnnnnnnnnnnnnn

SEQ ID NO: 9 - DNA, 120 bp, Artificial Sequence
  Synthetic construct
  misc_feature (19)..(38): n is a, c, g, or t
taatacgactcactatagnnnnnnnnnnnnnnnnnnnngttttagagctagaaatagcaagttaaaataaggctagtccgttatcaacttgaaaaagtggcaccgagtcggtgctttttt

SEQ ID NO: 10 - RNA, 24 nt, Artificial Sequence
  Synthetic construct
aauauaacuucguauaauguaugc

SEQ ID NO: 11 - RNA, 24 nt, Artificial Sequence
  Synthetic construct
uccaaacucaucaauguaucuuau

SEQ ID NO: 12 - RNA, 24 nt, Artificial Sequence
  Synthetic construct
cgucgccguccagcucgaccagga

SEQ ID NO: 13 - RNA, 20 nt, Artificial Sequence
  Synthetic construct
uaauuucuacucuuguagau

SEQ ID NO: 14 - RNA, 80 nt, Artificial Sequence
  Synthetic construct
guuuuagagcuagaaauagcaaguuaaaauaaggcuaguccguuaucaacuugaaaaaguggcaccgagucggugcuuuu

SEQ ID NO: 15 - DNA, 20 bp, Artificial Sequence
  Synthetic construct
ccttaatgcaccactcagca

SEQ ID NO: 16 - DNA, 20 bp, Artificial Sequence
  Synthetic construct
ggcttccactctctgaatgc

SEQ ID NO: 17 - DNA, 21 bp, Artificial Sequence
  Synthetic construct
tgagcaagggcgaggagctgt

SEQ ID NO: 18 - DNA, 24 bp, Artificial Sequence
  Synthetic construct
gtcctccttgaagtcgatgccctt

SEQ ID NO: 19 - DNA, 46 bp, Artificial Sequence
  Synthetic construct
tcttgtggaaaggacgaaacaccgtcatctgcaccaccggcaagct

SEQ ID NO: 20 - DNA, 48 bp, Artificial Sequence
  Synthetic construct
tctactattctttcccctgcactgtccgtcgtccttgaagaagatggt

SEQ ID NO: 21 - DNA, 48 bp, Artificial Sequence
  Synthetic construct
tcttgtggaaaggacgaaacaccgtgagcaagggcgaggagctgttca

SEQ ID NO: 22 - DNA, 43 bp, Artificial Sequence
  Synthetic construct
tctactattctttcccctgcactgtttgccgtaggtggcatcg

SEQ ID NO: 23 - DNA, 23 bp, Artificial Sequence
  Synthetic construct
gctgtgagataagagattcctgc

SEQ ID NO: 24 - DNA, 22 bp, Artificial Sequence
  Synthetic construct
actcccactatgggtttcttgg

SEQ ID NO: 25 - DNA, 23 bp, Artificial Sequence
  Synthetic construct
atcaacaagcaattcctcgatga

SEQ ID NO: 26 - DNA, 22 bp, Artificial Sequence
  Synthetiic construct
cagcattcgcttcaaggacata

SEQ ID NO: 27 - DNA, 22 bp, Artificial Sequence
  Synthetic construct
gagtatgacgattctgctgagg

SEQ ID NO: 28 - DNA, 21 bp, Artificial Sequence
  Synthetic construct
cagaccgaacgtgaagacgag

SEQ ID NO: 29 - DNA, 22 bp, Artificial Sequence
  Synthetic construct
gagctgtttgcagacaaagttc

SEQ ID NO: 30 - DNA, 20 bp, Artificial Sequence
  Synthetic construct
ccctggcacatgaatcctgg

SEQ ID NO: 31 - DNA, 28 bp, Artificial Sequence
  Synthetic construct
tccttctgttgattctgtcagtcctact

SEQ ID NO: 32 - DNA, 28 bp, Artificial Sequence
  Synthetic construct
agtaggactgacagaatcaacagaagga

SEQ ID NO: 33 - DNA, 23 bp, Artificial Sequence
  Synthetic construct
ccctcgtgaccaccttcggctac

SEQ ID NO: 34 - DNA, 23 bp, Artificial Sequence
  Synthetic construct
gtagccgaaggtggtcacgaggg

SEQ ID NO: 35 - DNA, 28 bp, Artificial Sequence
  Synthetic construct
tcctggtcgagctggacggcgacgtaaa

SEQ ID NO: 36 - DNA, 28 bp, Artificial Sequence
  Synthetic construct
tttacgtcgccgtccagctcgaccagga

SEQ ID NO: 37 - DNA, 26 bp, Artificial Sequence
  Synthetic construct
  misc_feature (1)..(1): 5′ thiol modifier C6 S-S
gaaatatgccagaaatatctgtcaga